(19) United States
(12) Patent Application Publication
(10) Pub. No.: US 2025/0304547 A1
(43) Pub. Date: Oct. 2, 2025

(54) Sulfur-Containing Lipids

(71) Applicant: NanoVation Therapeutics Inc., Vancouver (CA)

(72) Inventors: N. D. Prasad Atmuri, Vancouver (CA); Deaglan Arnold, London (CA); Fariba Saadati, Vancouver (CA); Marco A. Ciufolini, Vancouver (CA)

(73) Assignee: NanoVation Therapeutics Inc., Vancouver, BC (CA)

(21) Appl. No.: 18/864,520

(22) PCT Filed: May 11, 2023
(86) PCT No.: PCT/CA2023/050644

§ 371 Date: Nov. 9, 2024

Related U.S. Application Data
(60) Provisional application No. 63/340,687, filed on May 11, 2022.

(51) Int. Cl. C07D 317/28; C07C 323/12; C07C 323/25; C07D 317/24

(52) U.S. Cl. CPC C07D 317/28; C07C 323/12; C07C 323/25; C07D 317/24

(57) ABSTRACT

Provided herein are novel sulfur-containing lipids having a structure of Formula A or a salt thereof. The compounds may be formulated in a lipid nanoparticle for use in the delivery of charged cargo such as nucleic acids for use in the targeting of a non-liver organ, tissue or cell. Further provided are methods for making the compounds. (Formula A)

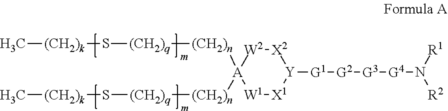

### INCORPORATION BY REFERENCE TO PRIORITY APPLICATION

This application claims priority from U.S. provisional application Ser. No. 63/340,687, filed on May 11, 2022, which is hereby expressly incorporated herein by reference in its entirety.

### TECHNICAL FIELD

Provided herein are lipids that may be formulated in a delivery vehicle so as to facilitate the encapsulation of a wide range of therapeutic agents or prodrugs therein, such as, without limitation, nucleic acids (e.g., RNA or DNA), proteins, peptides, pharmaceutical drugs and salts thereof.

### BACKGROUND

Nucleic acid-based therapeutics have enormous potential in medicine. To realize this potential, however, the nucleic acid must be delivered to a target site in a patient. This presents challenges since nucleic acid is rapidly degraded by enzymes in the plasma upon administration. Even if the nucleic acid is delivered to a disease site, there still remains the challenge of intracellular delivery. To address these problems, lipid nanoparticles have been developed that protect nucleic acid from such degradation and facilitate delivery across cellular membranes to gain access to the intracellular compartment, where the relevant translation machinery resides.

A key component of lipid nanoparticles (LNPs) is an ionizable lipid. The ionizable lipid is typically positively charged at low pH, which facilitates association with the negatively charged nucleic acid. However, the ionizable lipid is neutral at physiological pH, making it more biocompatible in biological systems. Further, it has been suggested that after the lipid nanoparticles are taken up by a cell by endocytosis, the ability of these lipids to ionize at low pH facilitates endosomal escape. This in turn enables the nucleic acid to be released into the intracellular compartment. While most research on cationic LNPs has focused on the formulation of nucleic acid, the delivery of other therapeutic agents or prodrugs besides nucleic acid is possible as well using the delivery platform.

Significant research has been devoted to identifying amino lipids with high potency. An ionizable lipid, referred to as DLin-MC3-DMA or “MC3” (dilinoleyl-methyl-4-dimethylaminobutyrate, 1), constitutes the state-of-the-art ionizable lipid for siRNA formulations. This ionizable lipid is a key component of Onpattro®, a lipid nanoparticle formulation incorporating siRNA that silences genes causing a genetic neurodegenerative disease referred to as hereditary transthyretin-mediated amyloidosis. Such formulations containing MC3 constituted the first small interfering RNA (siRNA) based treatments to be approved by the U.S. Food and Drug Administration (FDA).

The MC3 ionizable lipid is widely regarded as being an improved version of another amino lipid referred to as KC2, 2, being about 3 times more efficacious. In a study of over 50 amino lipids, MC3 was identified as having an ED₅₀ of 0.03 while that of KC2 was 0.10 for FVII gene silencing in mice using siRNA. (Jayaraman et al., 2012, Angew. Chem. Int. Ed., 51:8529-8533). This means that formulations containing MC3 require about 3 times less siRNA to attain the same end-result as similar formulations based on KC2. Since nucleic acid is costly, this translates into considerable savings for large scale manufacture of the ionizable lipid.

Ciufolini, et al., PCT/CA2022/050042 (filed on Jan. 12, 2022), teaches that a lipid termed MF19, 3, wherein sulfur atoms replace the C═C double bonds present in 1, is even more efficacious than MC3.

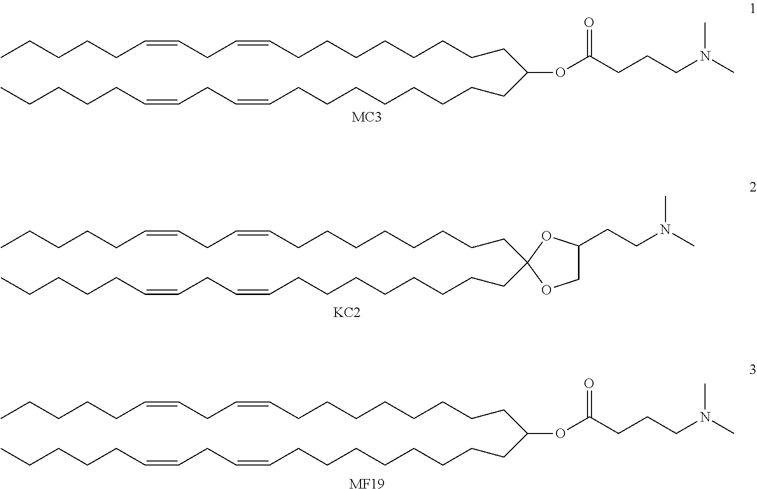

The above notwithstanding, there remains a need in the art for ionizable lipids for delivery of therapeutic agents or prodrugs that have a potency that is improved or comparable to known lipids, that are more organ-selective, and/or that can be manufactured conveniently or cost effectively.

### Definitions

As used herein, the term “ionizable lipid” refers to a lipid that, at a given pH, is in an electrostatically neutral form and that may either accept or donate protons, thereby becoming electrostatically charged, and for which the electrostatically neutral form has a calculated logarithm of the partition coefficient between water and 1-octanol (i.e., a cLogP) greater than 8.

As used herein, the term “MF19-type lipid” refers to any lipid, including but not limited to an ionizable lipid, of the type 3, and more generally described by the structure defined as Formula I below, with integer indices k independently ranging from 1 to 12; integer indices m independently ranging from 1 to 3; integer indices n independently ranging from 1 to 12; integer index p ranging from 1 to 6; integer indices q independently ranging from 1 to 6:

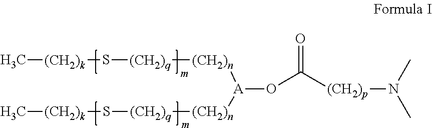

Lipids of the type shown as Formula I are described in WO 2022/155728, which is incorporated herein by reference.

As used herein, the term “alkyl” refers to a carbon-containing chain that is linear or branched, that may optionally have varying degrees of unsaturation, that may optionally incorporate N, O, and S atoms in the chain, and that may optionally exhibit substituents such another alkyl, OH, O-alkyl, O—Si(alkyl)3, S-alkyl.

As used herein, the term “lipophilic moiety” of an MF19-type lipid refers to the molecular portion of Formula I comprising the sulfur-containing alkyl group (“lipophilic chains”) and the carbon atom onto which they converge, but excluding the moiety, OOC—(CH₂)ₚ—NMe₂ bound to said carbon. Thus, in the case of Formula I, the lipophilic moiety is the one represented below as Formula II below and the lipophilic chains are the two groups represented as H₃C—(CH₂)ₖ[S—(CH₂)_{q}—]ₘ—(CH₂)ₙ, wherein integer indices k, m, n, and q are as defined above for Formula I:

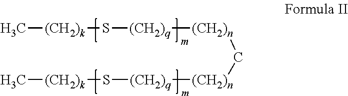

As used herein, the term “ionizable head” refers to the molecular moiety that is bound to the carbon atom onto which the sulfur-containing alkyl groups converge (“C” in Formula II above), said molecular moiety bearing the subunit capable of accepting or donating a proton, thereby becoming electrostatically charged. Thus, in the case of Formula I, the ionizable head is the one represented as Formula III below:

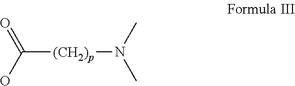

As used herein, the term “small alkyl” refers to a linear or branched carbon chain having a total of up to 6 carbon atoms, and that may be optionally unsaturated.

As used herein, the term “type 1 ionizable head” refers to a head group moiety of Formula IV, where integer index n ranges from 1 to 4, R¹ is H or a C₁-C₃ alkyl, R² is C₁-C₆ alkyl optionally bearing an OH substituent.

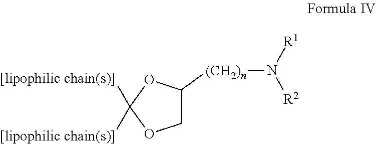

As used herein, the term “type 2 ionizable head” refers to a head group moiety of Formula V, where integer indices m and n range, independently, from 1 to 6, R¹ is H or a C₁-C₃ alkyl, R² is C₁-C₆ alkyl optionally bearing an OH substituent.

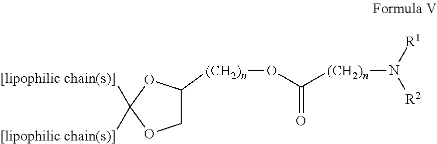

As used herein, the term “type 3 ionizable head” refers to a head group moiety of Formula VI, where integer index m ranges from 0 to 5, integer index n ranges from 1 to 6, R¹ is H or a C₁-C₃ alkyl, R² is C₁-C₆ alkyl optionally bearing an OH substituent.

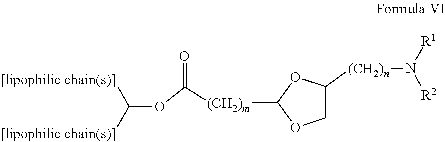

As used herein, the term “type 4 ionizable head” refers to a head group moiety of Formula VII, where R¹ is H or a C₁-C₃ alkyl, R² is C₁-C₆ alkyl optionally bearing an OH substituent or an NR³R⁴ substituent, wherein R³ is H or a C₁-C₃ alkyl and R⁴ is C₁-C₆ alkyl optionally bearing an OH substituent.

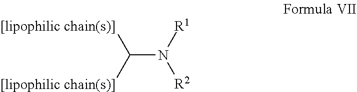

As used herein, the term “helper lipid” means a compound selected from: a sterol such as cholesterol or a derivative thereof; a diacylglycerol or a derivative thereof, such as a glycerophospholipid, including phosphatidic acid (phosphatidate) (PA), phosphatidylethanolamine (cephalin) (PE), phosphatidylcholine (PC), phosphatidylserine (PS), and the like; and a sphingolipid, such as a ceramide, a sphingomyelin, a cerebroside, a ganglioside, or reduced analogues thereof, that lack a double bond in the sphingosine unit. The term encompasses lipids that are either naturally-occurring or synthetic.

As used herein, the term “delivery vehicle” includes any preparation in which the lipid described herein is capable of being formulated and includes but is not limited to delivery vehicles comprising helper lipids.

As used herein, the term “nanoparticle” is any suitable particle in which the lipid can be formulated and that may comprise one or more helper lipid components. The one or more lipid components may include an ionizable lipid prepared by the method described herein and/or may include additional lipid components, such as a helper lipid. The term includes, but is not limited to, vesicles with one or more bilayers, including multilamellar vesicles, unilamellar vesicles and vesicles with an electron-dense core. The term also includes polymer-lipid hybrids, including particles in which the lipid is attached to a polymer.

As used herein, the term “encapsulation,” with reference to incorporating a cargo molecule within a delivery vehicle refers to any association of the cargo with any component or compartment of the delivery vehicle such as a nanoparticle.

### SUMMARY

In some embodiments, the present disclosure is based, in part, on the discovery that a delivery vehicle comprising certain sulfur-containing ionizable lipids exhibits surprising extrahepatic organ selectivity relative to an otherwise identical nanoparticle containing an MC3-type lipid without the sulfur groups.

To illustrate, and without intending to be limiting, MC3-type lipids and sulfur-containing ionizable lipids that are described by Formula A herein typically have comparable physicochemical properties (pKa values, percent ofnucleic acid entrapment, polydispersity index (PDI) and the like). However, it has been found that otherwise identical formulations of nucleic acid incorporating the sulfur-containing ionizable lipid of the disclosure or an MC3 lipid vary in their selectivity for the spleen. In vivo studies in examples of the disclosure demonstrate that delivery vehicles incorporating the ionizable sulfur lipids herein deliver nucleic acid cargo at significantly higher levels to the spleen relative to the same delivery vehicle incorporating an MC3-type lipid. This may translate into considerably improved efficacy in extrahepatic tissues. For example, in some embodiments, a smaller dose of nucleic acid encapsulated in an LNP having the sulfur-lipids described herein may achieve the same response in the spleen relative to formulations comprising known ionizable lipids, such as MC3. Due to the high cost of ionizable lipid, embodiments of the disclosure could provide for considerable cost savings for preparing delivery vehicles comprising ionizable lipid.

Furthermore, the inventors have discovered that, in some embodiments, the ionizable lipids of the disclosure may be readily and economically prepared by Claisen technology described in co-pending and co-owned WO 2022/246555, titled “Method for Producing an Ionizable Lipid”, which is incorporated herein by reference.

Other objects, features, and advantages of the present disclosure will be apparent to those of skill in the art from the following detailed description and figures.

### BRIEF DESCRIPTION OF THE DRAWINGS

FIG. 1A is a bar graph showing entrapment (%), particle size (nm) and polydispersity index (PDI) of firefly luciferase mRNA-containing lipid nanoparticles (LNPs) comprising the ionizable lipid nor-MC3 as described in WO 2022/246571 (incorporated herein by reference) or 4 (described herein). The LNPs are composed of 50/10/38.5/1.5 mol % of ionizable lipid/DSPC/chol/PEG-DMG and the amine-to-phosphate (N/P) was 6.

FIG. 1B shows luminescence intensity/mg in the liver (left graph) or spleen (right graph) for the mRNA-containing LNPs comprising the ionizable lipid nor-MC3 or 4 at 4 hours post-intravenous administration to CD-1 mice. The LNPs contain 50/10/38.5/1.5 mol % of ionizable lipid/DSPC/chol/PEG-DMG (N/P=6).

### DETAILED DESCRIPTION

Embodiments disclosed herein relate to lipids having the structure of Formula A:

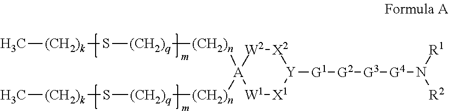

or a salt thereof,
wherein:
integer indices k independently range from 1 to 10,
integer indices m independently range from 1 to 4,
integer indices n independently range from 1 to 10
integer indices q independently range from 1 to 5
A is either C or N, and
   If A is C, then
   W¹ and X¹ are either bonded to each other or not bonded to each other (as indicated by the dashed bond), and
      if W¹ and X¹ are bonded to each other, then
         W¹ may be O or S;
         W² may be 0 or S;
         X¹ is (CH₂)ₘ, with n ranging from 1 to 2;
         X² is (CH₂)ₙ, with n ranging from 0 to 2;
         Y is CH
         G¹ is (CH₂)ᵣ, with r ranging from 1 to 6;
         Groups G²-G³-G⁴ are either present or absent, and:
         if G²-G³-G⁴ are present, then:
         G² is O;
         G³ is a carbonyl (i.e., a C═O) group;
         G⁴ is (CH₂)ₛ, with s ranging from 1 to 6;
         R¹ is a C₁-C₃ alkyl
         R² is a C₁-C₆ alkyl optionally bearing an OH substituent
         if G²-G³-G⁴ are absent, then the moiety NR¹R² is directly bonded to G¹ and
         R¹ is a C₁-C₃ alkyl and
         R² is a C₁-C₆ alkyl optionally bearing an OH substituent.
      If W¹ and X¹ are not bonded to each other, then:
         W¹ is H;
         W² may be absent or it may be present, and
         If W² is absent, then
         X¹, X², Y, and moiety G¹-G²-G³-G⁴ are all absent and
         NR¹R², wherein R¹ is a C₁-C₃ alkyl and R² is a C₁-C₆ alkyl optionally bearing an OH substituent, is bound directly to the C atom bearing W¹.
         If W² is present, then
         W² is O or N, and
         if W² is 0, then
         X¹—Y is a carbonyl (C═O) group, with Y being the carbonyl carbon and X¹ being the carbonyl oxygen, and with X¹ and Y sharing a double bond of the carbonyl group;
         X² is absent, and Y is singly bonded to W²;
         G¹ is (CH₂)ₜ, with t ranging from 1 to 6;
         groups G²-G³-G⁴ are either absent or present, and:
         if G²-G³-G⁴ are absent, then
         NR¹R², wherein R¹ is a C₁-C₃ alkyl and R² is a C₁-C₆ alkyl bearing an OH substituent, is bound directly to G¹
         if G²-G³-G⁴ are present, then
         G² is the structure shown below, bonded to G¹ and to
         G³ as indicated (G¹ is bonded to position 2 of G² and
         G³ is bonded to position 4 of G²), and wherein E is O or S:

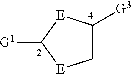

G³ is (CH₂)ᵤ, with u=1 3;
G⁴ is (CH₂)ᵥ, with v=1 3;
R¹ is a C₁-C₃ alkyl and R² is a C₁-C₆ alkyl optionally bearing an OH substituent.
if W² is N, then
X¹, X², Y, G¹, G², G³ are absent and W² is bonded to G⁴
G⁴ is (CH₂)ₜ, with t ranging from 1 to 6;
R¹ is a C₁-C₃ alkyl and R² is a C₁-C₆ alkyl optionally bearing an OH substituent.
If A is N, then
   the segment, W², X¹, X², Y, G¹, G², G³, G⁴-NR¹R² is absent, and
   W¹ is (CH₂)ₜ—OH, with t ranging from 1 to 6.

The salt includes any pharmaceutically acceptable salt known to those of ordinary skill in the art.

### Methods to Produce the Lipids of the Disclosure

Representative, but by no means limiting, examples of lipids possessing the structure of Formula A are compounds 4-17 below:

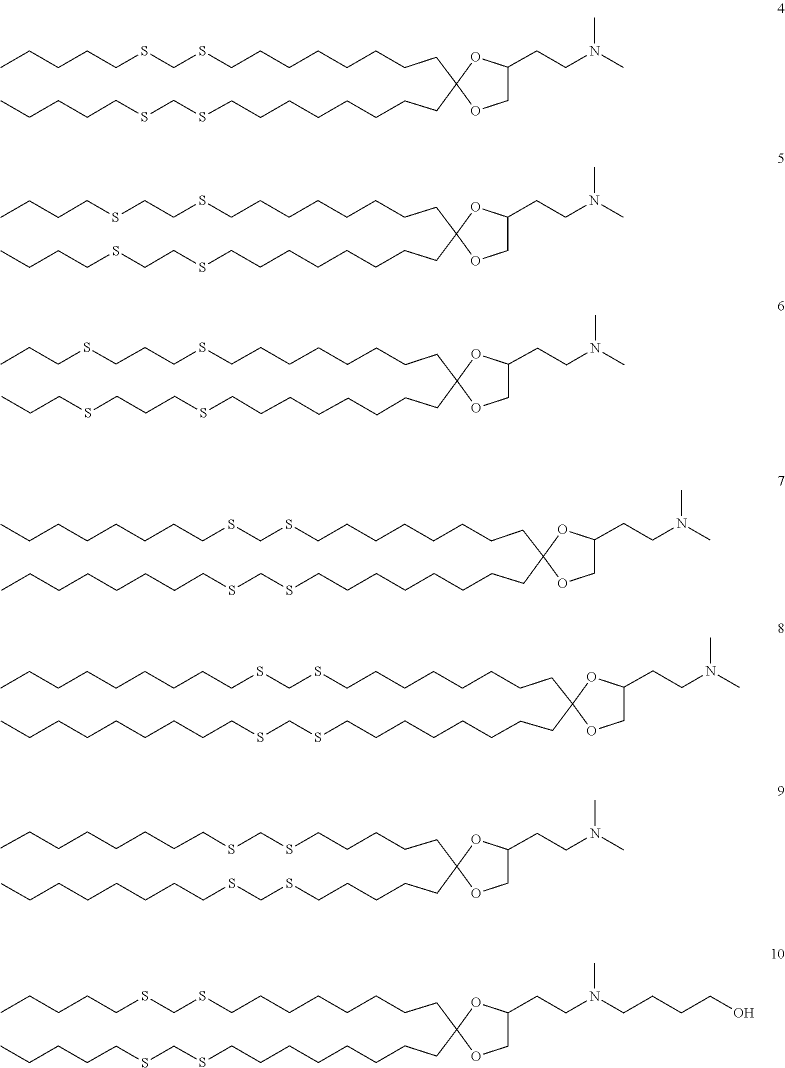

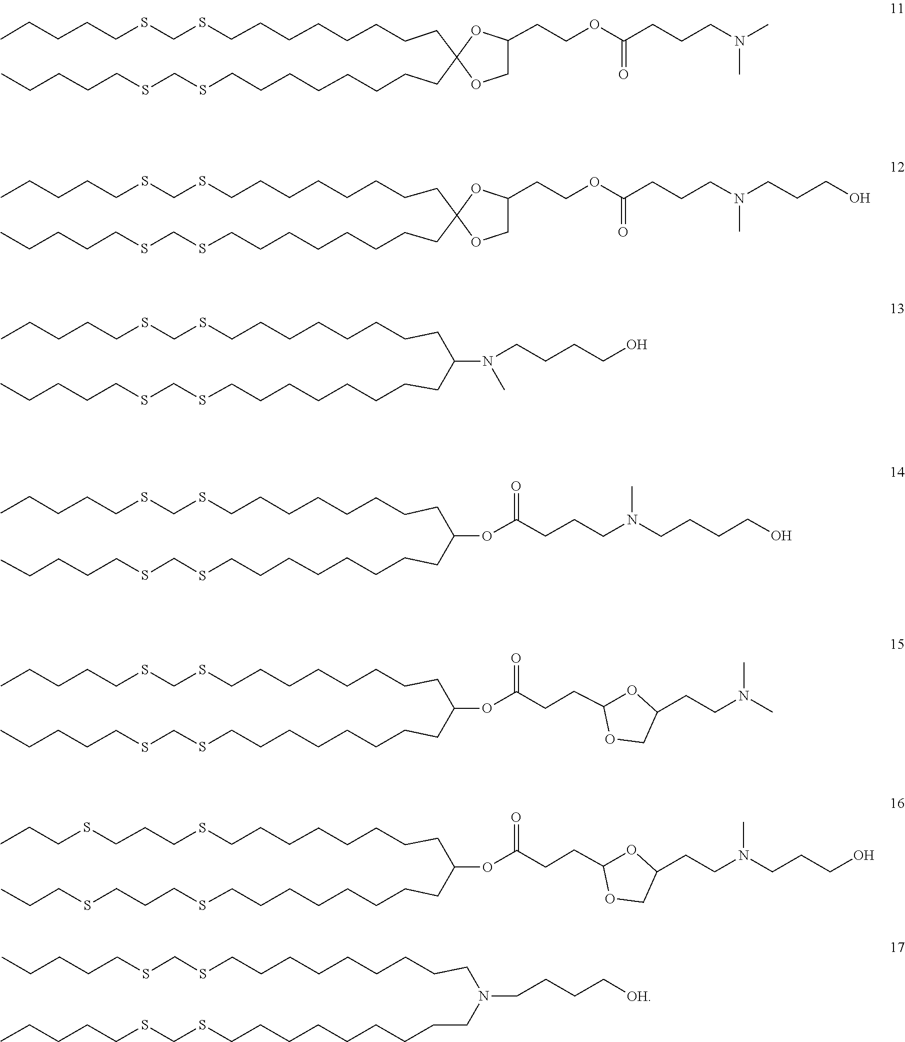

Lipids having the structure of Formula A wherein A is C, and comprising an ionizable head group of type 1-4, such as compounds 4-16, can be prepared from ketone intermediates of general formula 20 and/or alcohol intermediates of general formula 21 (Scheme 1).

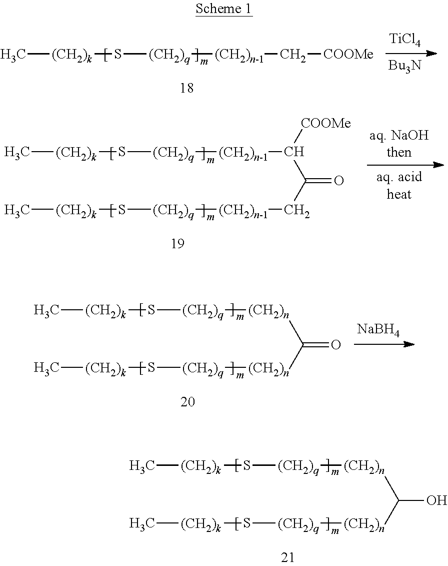

Compounds 20-21 can be manufactured starting with a Claisen condensation of esters of general formula 18, as described in co-pending and co-owned WO 2022/246555, which is incorporated herein by reference.

Esters 18 can be made starting from appropriate o-hydroxyesters such as 24 (Scheme 2). The latter can be prepared starting with, for example, a selective hydrolysis of a dicarboxylic acids diester, e.g., methyl ester 22, to monoester 23, for example by treatment with methanolic Ba(OH)₂ followed by aqueous acid (Vozdvizhenskaya, O. A.; et al. Chem. Het. Comp. 2021, 57, 490; incorporated herein by reference). Subsequently, the COOH group in monoesters 23 is selectively reduced, for example with borane-dimethyl sulfide complex or borane-THF complex, to produce 24. Alternatively, o-hydroxyesters 24 can be made by subjecting a lactone such as 26 to methanolysis, for example, by treatment with methanol and K₂CO₃. In some embodiments, lactones 26 are manufactured by Baeyer-Villiger oxidation of ketones 25, as described in a co-owned and co-pending PCT application (PCT/CA2023/050129, incorporated herein by reference). Finally, the OH group in 24 is converted into a good leaving group, for example a sulfonate ester such as mesylate 27.

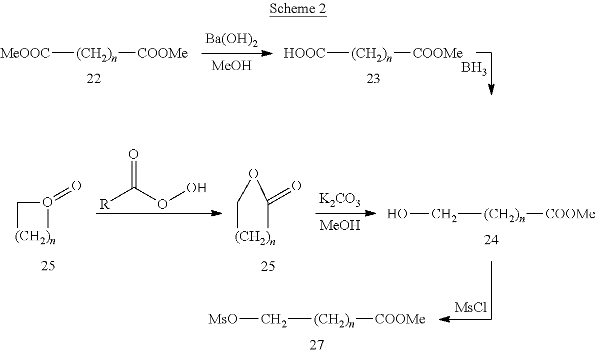

Compounds of the type 18 with m=1 and q=1, that is, substances 28, can be obtained from 27 by reaction with a thiol under basic conditions, as indicated in Scheme 3:

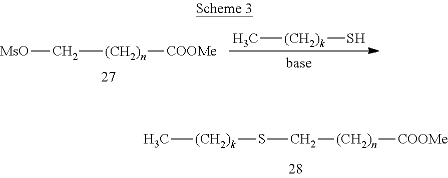

Compounds of the type 18 with m=2 and q=1, that is, substances 30, can be obtained from 27 and as indicated in Scheme 4:

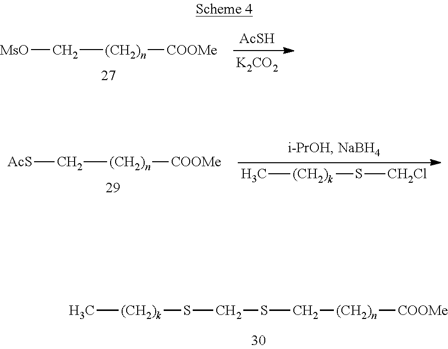

Compounds of the type 18 with m=2 and q=2, that is, substances 33, can be obtained from 27 as indicated in Scheme 5:

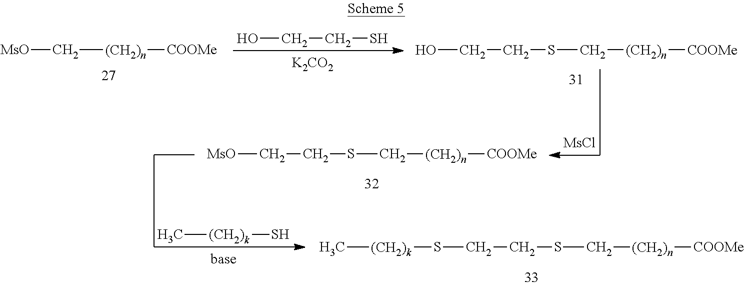

Compounds of the type 18 with m=2 and q=3, that is, substances 36, can be obtained from 27 as indicated in Scheme 6:

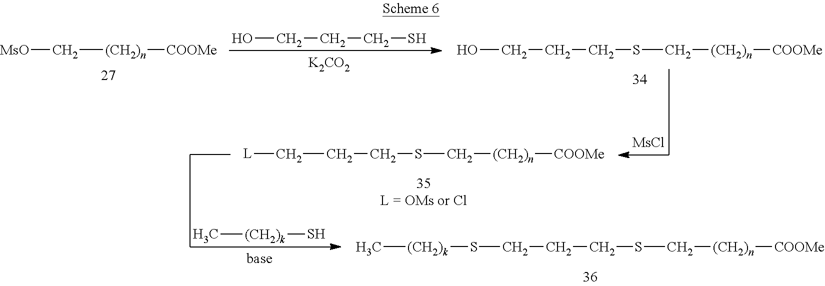

Compounds of the type 18 with other values of m and q can be obtained by modification of synthetic Schemes 3-6 above in a manner that would be evident to those skilled in the art.

Without intending to be limiting, the diagrams below illustrate how the chemistry of Schemes 1-6 above can be used to prepare ketones 54-56 (Schemes 7-11) and alcohols 75-76 (Scheme 19) from dimethyl azelate and convert them into appropriate ionizable lipids through the attachment of a suitable ionizable head group.

However, the diagrams below are by no means to be construed as implying that the methodology is limited to azelaic esters. Azelaic ester was chosen only to (i) exemplify the synthetic methods disclosed herein, (ii) simplify the presentation of representative synthetic routes, and (iii) illustrate the diversity of final products that are available from a single early intermediate.

Thus, dimethyl azelate, 37 is transformed into mesylate 40 by the method set forth earlier in Scheme 2 (Scheme 7).

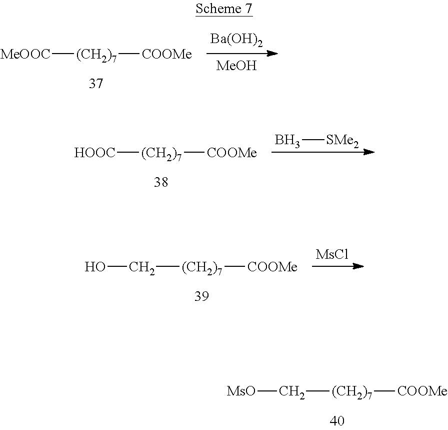

Mesylate 40 can be transformed into an ester of the type 18 with m=2 and q=1, such as compound 44, as indicated in Scheme 8.

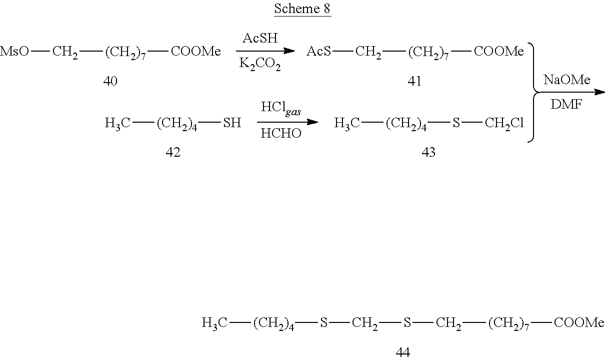

Mesylate 40 can be transformed into an ester of the type 18 with m=2 and q=2, that is, substance 47, as indicated in Scheme 9:

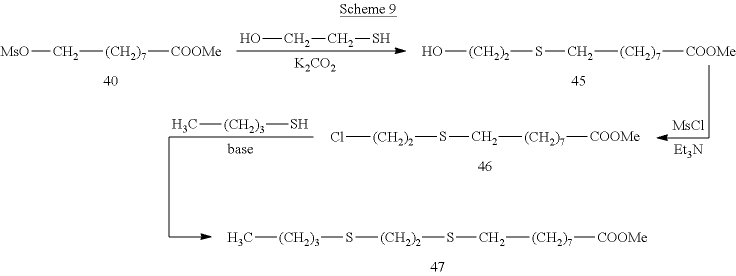

Mesylate 40 can be transformed into an ester of the type 18 with m=2 and q=3, that is, substance 50, as indicated in Scheme 10:

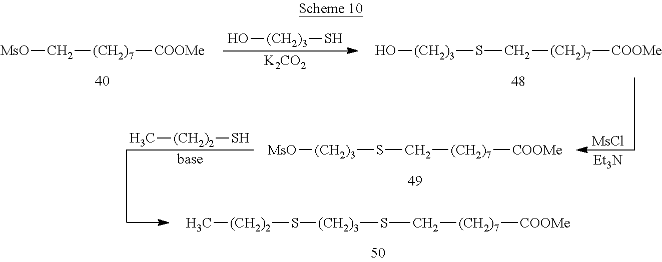

The Claisen condensation of esters 44, 47 and 50, as described in co-owned and co-pending WO 2022/246555 (incorporated herein by reference), produces ketoesters 51, 52 and 53, respectively (Scheme 11). These

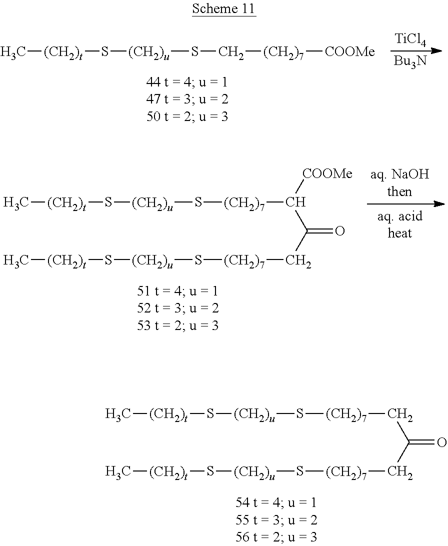

are transformed into ketones 54, 55 and 56, respectively, by hydrolysis and decarboxylation. Any of the lipids possessing a structure of Formula A, wherein A is C, can be prepared from ketones of the type 54-56 by converting the ketone into a head group of type 1-4.

For example, the synthesis of lipids 4-6 can be achieved as shown in Scheme 12. Ketalization of 54-56 with 1,2,4-butanetriol, for example in refluxing toluene in the presence of an acid catalyst, for example, pyridinium para-toluenesulfonate (PPTS), and with continuous removal of water, for example, by the use of a Dean-Stark trap, produces ketals 57-59. The OH group in the latter is transformed into a leaving group, for example a sulfonate such as a mesylate, to give compounds 60-62. Reaction of the latter mesylates with dimethylamine in an appropriate solvent at a temperature between 0 and 150° C., optionally with microwave irradiation (Buschmann, M. D., et al., Commun. Biol. 2021, 4, 956; https://doi.org/10.1038/s42003-021-02441-2), produces lipids 4-6.

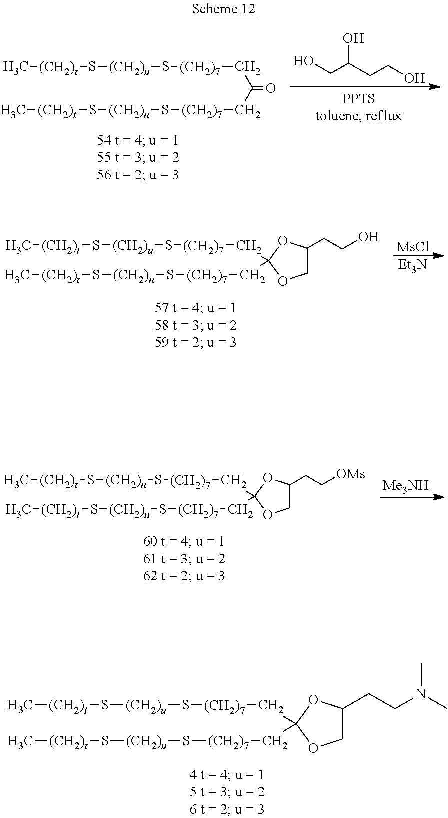

The synthesis of a lipid such as 10 can be achieved by reacting mesylate 60 with 4-(methylamino)-1-butanol in an appropriate solvent at a temperature between 0 and 150° C., optionally with microwave irradiation (Scheme 13).

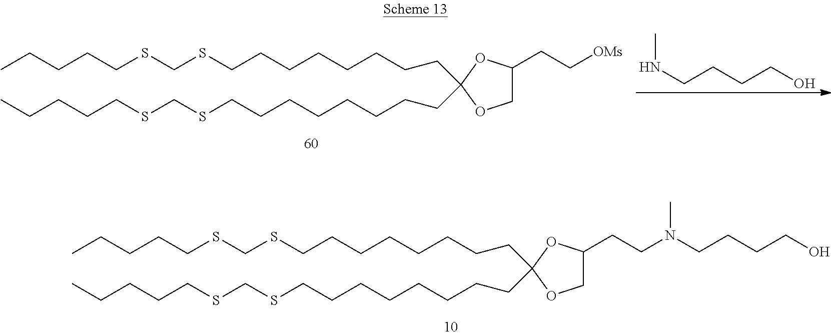

The synthesis of a lipid such as 11 can be achieved by coupling ketal 57 with 4-(dimethylamino)-butanoic acid, typically as its hydrochloride salt, in the presence of a condensing agent; for example, a carbodiimide such as EDCI (Scheme 14).

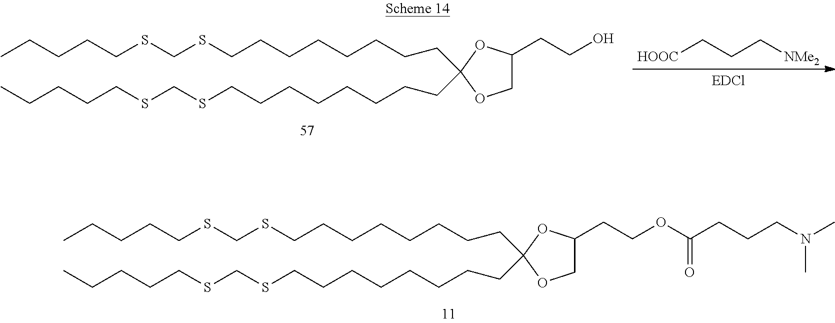 [SMILES: CCCCCSCSCCCCCCCCC1(CCCCCCCCSCSCCCCC)OCC(CCO)O1 ; CN(C)CCCC(=O)O ; CCCCCSCSCCCCCCCCC1(CCCCCCCCSCSCCCCC)OCC(CCOC(=O)CCCN(C)C)O1]

The synthesis of a lipid such as 12 can be achieved by coupling ketal 57 with, for example, 4-((3-((tert-butyldimethylsilyl)oxy)propyl)(methyl)amino)butanoic acid (the preparation of which is provided in the experimental section) or its HCl salt, in the presence of a condensing agent; for example, a carbodiimide such as EDCI, followed by release of the silyl protecting group, for example with a source of fluoride ion such as pyridine-HF complex (Scheme 15).

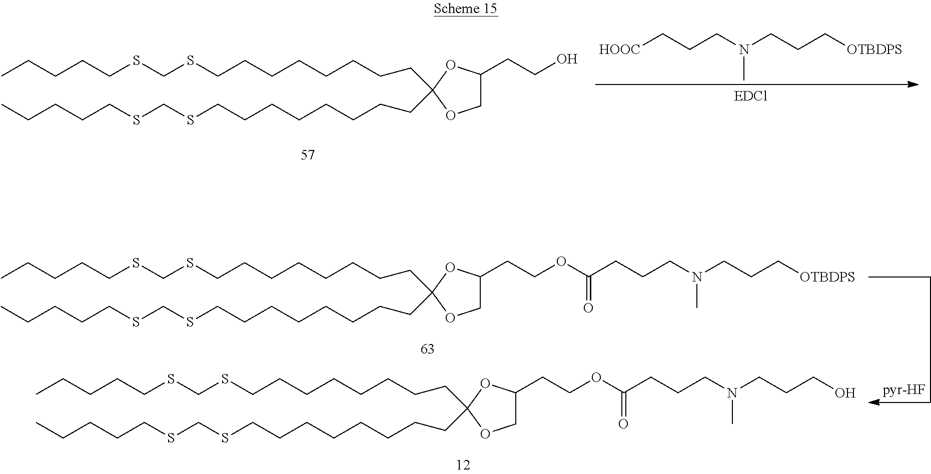

The synthesis of a lipid such as 13 can be achieved by reductive amination of ketone 54 with, for example, the tert-butyldiphenylsilyl ether of 4-amino-1-butanol in the presence of a hydride donor; for example, a boron hydride such as sodium triacetoxyborohydride, sodium cyanoborohydride, and the like, and optionally in the presence of a weak acid such as acetic acid. Amine 64 thus formed is further N-methylated, for example, with aqueous formaldehyde in the presence of a hydride donor; for example, a boron hydride such as sodium triacetoxyborohydride, sodium cyanoborohydride, and the like, to produce 65, which is transformed into 13 by release of the silyl protecting group, for example with a source of fluoride ion such as pyridine-HF complex (Scheme 16).

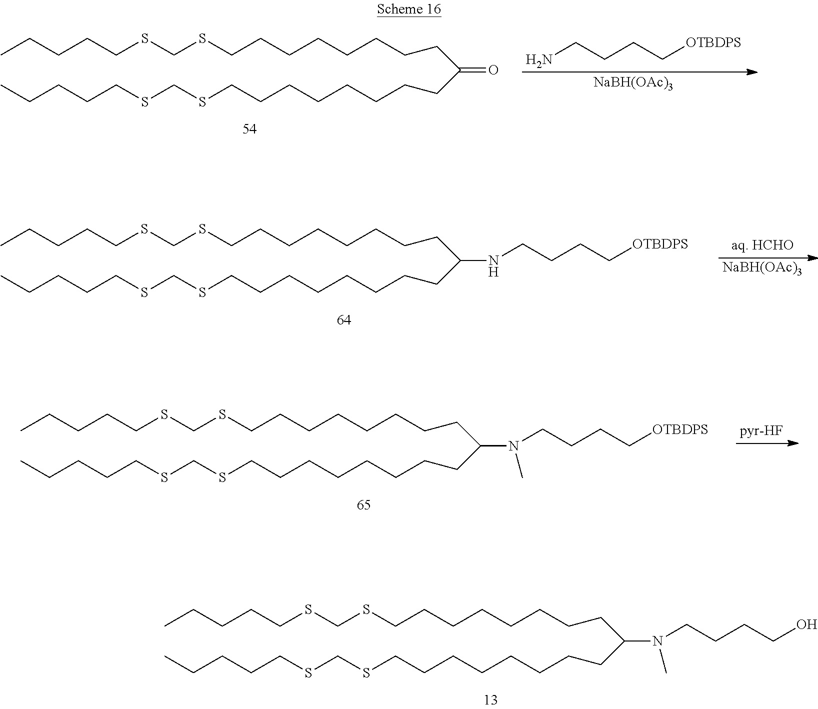

Lipids such as 7 and 8 can be prepared by modifications of the methods outlined above that would be obvious to the skilled artisan (Scheme 17).

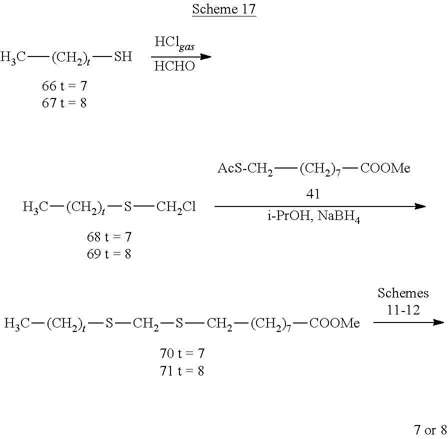

Lipids such as 9 can be prepared by a method analogous to that outlined in Schemes 10-12 above, but starting with monomethyl adipate (Scheme 18).

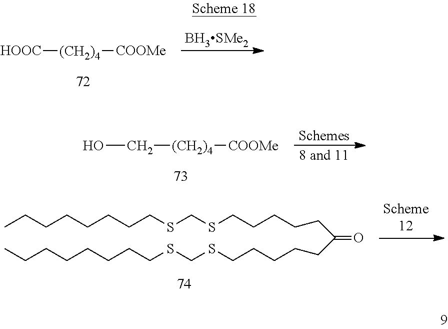

The synthesis of lipids such as 14-16 starts with the reduction of ketones 54 and 56 to the corresponding alcohols, 75 and 76, for example with a hydride reagent such as sodium borohydride (Scheme 19).

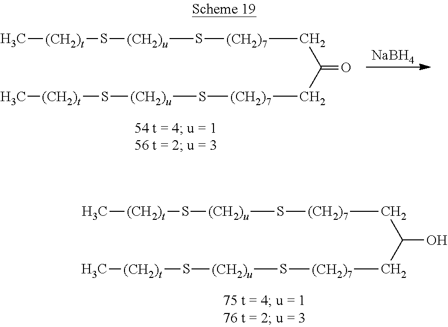

Esterification of 75 with 4-((4-((tert-butyldiphenylsilyl)oxy)butyl)(methyl)amino)butanoic acid (the preparation of which is provided in the experimental section) or its HCl salt, in the presence of a condensing agent, for example, a carbodiimide such as EDCI, followed by desilylation of the product with a source of fluoride ion, for example, pyr-HF, produces 14 (Scheme 20).

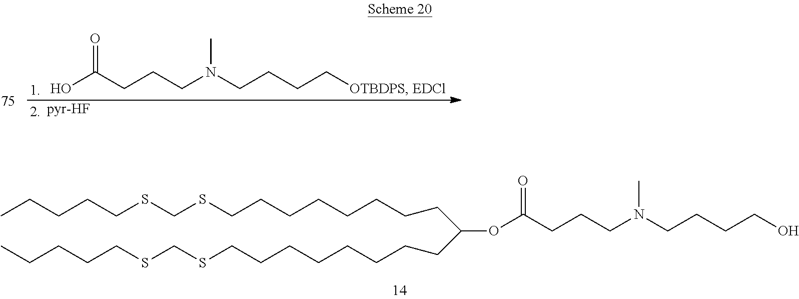

Esterification of 75 with 3-(4-(2-(dimethylamino)ethyl)-1,3-dioxolan-2-yl)propanoic acid (the preparation of which is provided in the experimental section) or its HCl salt, in the presence of a condensing agent, for example, a carbodiimide such as EDCI, produces 15 (Scheme 21).

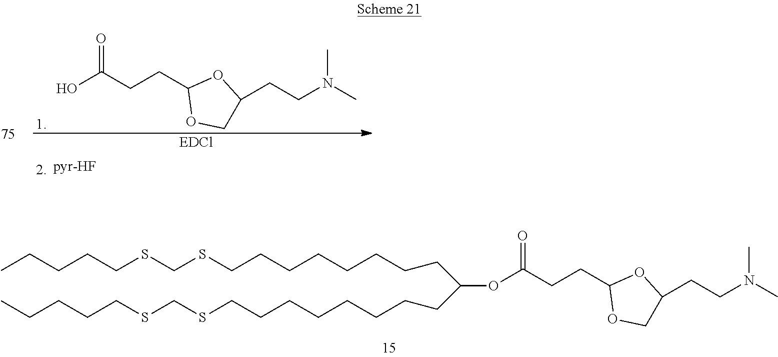

Esterification of 3-(4-(2-((3-((tert-butyldiphenylsilyl)oxy)propyl)(methyl)amino)ethyl)-1,3-dioxolan-2-yl)propanoic acid (the preparation of which is provided in the experimental section) or its HCl salt with 76 in the presence of a condensing agent, for example, a carbodiimide such as EDCI, followed by desilylation with a source of fluoride ion, for example, pyr-HF, produces 16 (Scheme 22).

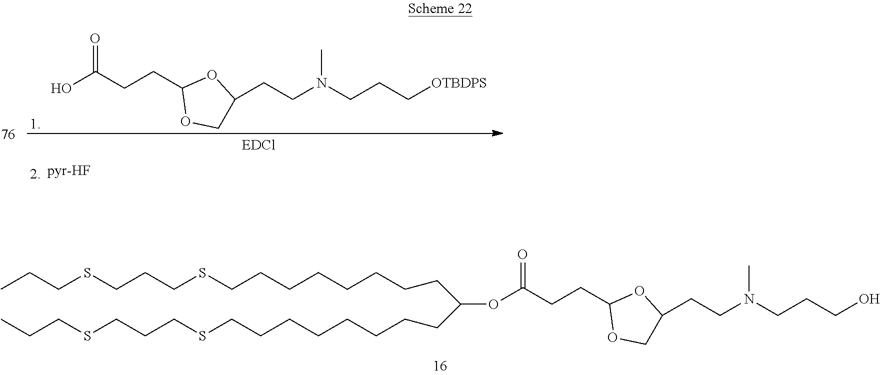

Lipids possessing the structure of Formula A wherein A is N, such as compound 14, can be prepared from ester intermediates of general formula 18 starting with reduction to alcohols of general formula 77, for example with lithium aluminum hydride (LAH, Scheme 22). The OH group in 77 is converted into a leaving group, for example, a sulfonate such as a mesylate or a tosylate, or a halide such as a chloride, bromide, or iodide, and the resulting 78 is employed to N-alkylate a primary amine as described in co-pending and co-owned PCT/CA2023/050287 filed on Mar. 6, 2023, which is incorporated herein by reference. This leads to lipids of general structure 79.

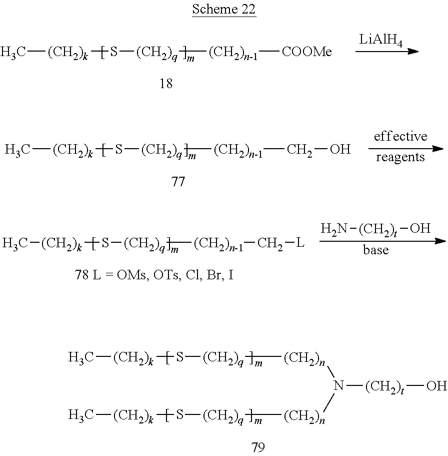

To exemplify, the synthesis of lipid 17 can be achieved starting with the reduction of ester 44 to alcohol 80 with a hydride reagent, for example, lithium aluminum hydride, followed by conversion of 80 into mesylate 81. The reaction 4-aminobutanol with 81 in acetonitrile at a temperature between 5° and 100° C. and in the presence of a base, for example, sodium carbonate, produces 17.

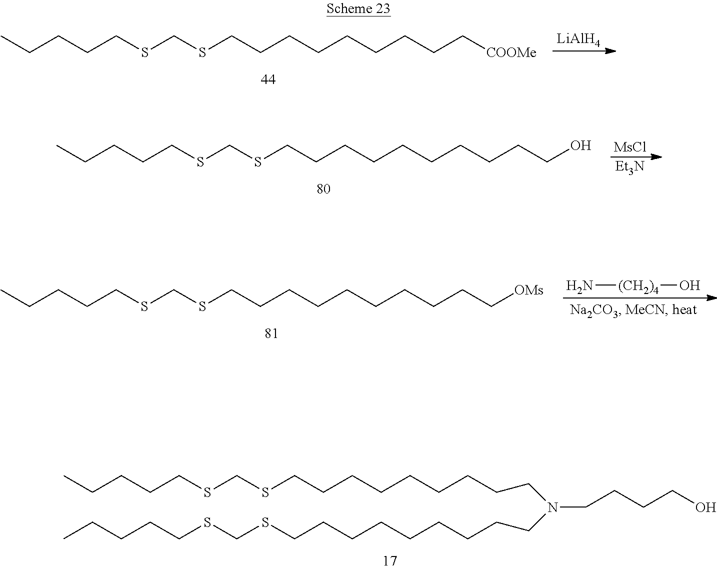

### Formulation of the Lipid in a Delivery Vehicle

The ionizable lipid of the disclosure may be formulated in a variety of drug delivery vehicles (also referred to herein as a “delivery vehicle”) known to those of ordinary skill in the art. An example of a delivery vehicle is a lipid nanoparticle, which includes liposomes, lipoplexes, polymer nanoparticles comprising lipids, polymer-based nanoparticles, emulsions, and micelles.

In one embodiment, the ionizable lipid of the disclosure is formulated in a delivery vehicle by mixing the ionizable lipid with additional lipids, including helper lipids, such as vesicle forming lipids and optionally an aggregation inhibiting lipid, such as a hydrophilic polymer-lipid conjugate (e.g., PEG-lipid).

As set forth previously, a helper lipid includes a sterol, a diacylglycerol, a ceramide or derivatives thereof.

Examples of sterols include cholesterol, or a cholesterol derivative, such as cholestanol, cholestanone, cholestenone, coprostanol, cholesteryl-2′-hydroxyethyl ether, cholesteryl-4′-hydroxybutyl ether, beta-sitosterol, fucosterol, and the like.

Examples of diacylglycerols include dipalmitoylphosphatidylcholine (DPPC), distearoylphosphatidylcholine (DSPC), dioleoylphosphatidylethanolamine (DOPE), palmitoyloleoyl-phosphatidylcholine (POPC), palmitoyloleoyl-phosphatidylethanolamine (POPE), palmitoyloleyol-phosphatidylglycerol (POPG), dipalmitoyl-phosphatidylethanolamine (DPPE), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), stearoyloleoyl-phosphatidylethanolamine (SOPE), egg phosphatidylcholine (EPC), and mixtures thereof. In certain embodiments, the phospholipid is DPPC, DSPC, or mixtures thereof. These lipids may be synthesized or obtained from natural sources, such as from egg.

A suitable ceramide derivative is egg sphingomyelin or dihydrosphingomyelin.

Delivery vehicles incorporating the ionizable lipids of the disclosure can be prepared using a wide variety of well described formulation methodologies known to those of skill in the art, including but not limited to extrusion, ethanol injection and in-line mixing. In one embodiment, the preparation method is an in-line mixing technique in which aqueous and organic solutions are mixed using a rapid-mixing device as described in Kulkari et al., 2018, ACS Nano, 12:4787 and Kulkarni et al., 2017, Nanoscale, 36:133347, each of which is incorporated herein by reference in its entirety.

The delivery vehicle can also be a nanoparticle that is a lipoplex that comprises a lipid core stabilized by a surfactant. Vesicle-forming lipids may be utilized as stabilizers. The lipid nanoparticle in another embodiment is a polymer-lipid hybrid system that comprises a polymer nanoparticle core surrounded by stabilizing lipid.

Nanoparticles comprising the ionizable lipid of the disclosure may alternatively be prepared from polymers without lipids. Such nanoparticles may comprise a concentrated core of a therapeutic agent that is surrounded by a polymeric shell or may have a solid or a liquid dispersed throughout a polymer matrix.

The ionizable lipids described herein can also be incorporated into emulsions, which are drug delivery vehicles that contain oil droplets or an oil core. An emulsion can be lipid-stabilized. For example, an emulsion may comprise an oil filled core stabilized by an emulsifying component such as a monolayer or bilayer of lipids.

The ionizable lipid may be incorporated into a micelle. Micelles are self-assembling particles composed of amphipathic lipids or polymeric components that are utilized for the delivery of agents present in the hydrophobic core.

A further class of drug delivery vehicles known to those of skill in the art that can be used to formulate the ionizable lipid herein is a carbon nanotube.

### Delivery of Nucleic Acid, Genetic Material, Proteins, Peptides or Other Charged Agents

The ionizable lipid disclosed herein may facilitate the incorporation of a compound or molecule (referred to herein also as “cargo” or “cargo molecule”) bearing a net negative or positive charge into the delivery vehicle and subsequent delivery to a target cell in vitro or in vivo.

In one embodiment, the cargo molecule is genetic material, such as a nucleic acid. The nucleic acid includes, without limitation, RNA, including small interfering RNA (siRNA), small nuclear RNA (snRNA), micro RNA (miRNA), messenger RNA (mRNA) or DNA such as vector DNA or linear DNA. The nucleic acid length can vary and can include nucleic acid of 5-50,000 nucleotides in length. The nucleic acid can be in any form, including single stranded DNA or RNA, double stranded DNA or RNA, or hybrids thereof. Single stranded nucleic acid includes antisense oligonucleotides.

In one embodiment, the cargo is an mRNA, which includes a polynucleotide that encodes at least one peptide, polypeptide or protein. The mRNA includes, but is not limited to, small activating RNA (saRNA) and trans-amplifying RNA (taRNA), as described in co-pending PCT/CA2023/050439, titled “mRNA Delivery Using Lipid Nanoparticles”, which is incorporated herein by reference.

The mRNA as used herein encompasses both modified and unmodified mRNA. In one embodiment, the mRNA comprises one or more coding and non-coding regions. The mRNA can be purified from natural sources, produced using recombinant expression systems and optionally purified, or may be chemically synthesized.

In those embodiments in which an mRNA is a chemically synthesized molecule, the mRNA can comprise nucleoside analogs such as analogs having chemically modified bases or sugars, and/or backbone modifications. In some embodiments, an mRNA is or comprises natural nucleosides (e.g., adenosine, guanosine, cytidine, uridine); nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, 2-thiocytidine, pseudouridine, and 5-methylcytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

The mRNA of the disclosure may be synthesized according to any of a variety of known methods. For example, mRNA in certain embodiments may be synthesized via in vitro transcription (IVT). Briefly, IVT is typically performed with a linear or circular DNA template containing a promoter, a pool of ribonucleotide triphosphates, a buffer system that may include DTT and magnesium ions, and an appropriate RNA polymerase (e.g., T3, T7 or SP6 RNA polymerase), DNAse I, pyrophosphatase, and/or RNAse inhibitor.

In some embodiments, in vitro synthesized mRNA may be purified before encapsulation to remove undesirable impurities including various enzymes and other reagents used during mRNA synthesis.

The present disclosure may be used to encapsulate mRNAs of a variety of lengths. In some embodiments, the present disclosure may be used to encapsulate in vitro synthesized mRNA ranging from about 1-20 kb, about 1-15 kb, about 1-10 kb, about 5-20 kb, about 5-15 kb, about 5-12 kb, about 5-10 kb, about 8-20 kb, or about 8-15 kb in length.

Typically, mRNA synthesis includes the addition of a “cap” on the 5′ end, and a “tail” on the 3′ end. The presence of the cap provides resistance to nucleases found in most eukaryotic cells. The presence of a “tail” serves to protect the mRNA from exonuclease degradation.

In some embodiments, mRNAs include a 5′ and/or 3′ untranslated region. In some embodiments, a 5′ untranslated region includes one or more elements that affect an mRNA's stability or translation, for example, an iron responsive element. In some embodiments, a 5′ untranslated region may be between about 50 and 500 nucleotides in length.

In some embodiments, a 3′ untranslated region includes one or more of a polyadenylation signal, a binding site for proteins that affect an mRNA's stability of location in a cell, or one or more binding sites for miRNAs. In some embodiments, a 3′ untranslated region may be between 50 and 500 nucleotides in length or longer.

While mRNA provided from in vitro transcription reactions may be desirable in certain embodiments, other sources of mRNA are contemplated, such as mRNA produced from bacteria, fungi, plants, and/or animals.

The mRNA sequence may comprise a reporter gene sequence, although the inclusion of a reporter gene sequence in pharmaceutical formulations for administration is optional. Such sequences may be incorporated into mRNA for in vitro studies or for in vivo studies in animal models to assess biodistribution.

In another embodiment, the cargo is an siRNA. An siRNA becomes incorporated into endogenous cellular machineries to result in mRNA breakdown, thereby preventing transcription. Since RNA is easily degraded, its incorporation into a delivery vehicle can reduce or prevent such degradation, thereby facilitating delivery to a target site. The siRNA encompassed by embodiments of the disclosure may be used to specifically inhibit expression of a wide variety of target polynucleotides. The siRNA molecules targeting specific polynucleotides may be readily prepared according to procedures known in the art. An siRNA target site may be selected and corresponding siRNAs may be chemically synthesized, created by in vitro transcription, or expressed from a vector or PCR product. A wide variety of different siRNA molecules may be used to target a specific gene or transcript. The siRNA may be double-stranded RNA, or a hybrid molecule comprising both RNA and DNA, e.g., one RNA strand and one DNA strand. The siRNA may be of a variety of lengths, such as 15 to 30 nucleotides in length or 20 to 25 nucleotides in length. In certain embodiments, the siRNA is double-stranded and has 3′ overhangs or 5′ overhangs. In certain embodiments, the overhangs are UU or dTdT 3′. In particular embodiments, the siRNA comprises a stem loop structure.

In a further embodiment, the cargo molecule is a microRNA or small nuclear RNA. Micro RNAs (miRNAs) are short, noncoding RNA molecules that are transcribed from genomic DNA, but are not translated into protein. These RNA molecules are believed to play a role in regulation of gene expression by binding to regions of target mRNA. Binding of miRNA to target mRNA may downregulate gene expression, such as by inducing translational repression, deadenylation or degradation of target mRNA. Small nuclear RNA (snRNA) are typically longer noncoding RNA molecules that are involved in gene splicing. The snRNA molecules may have therapeutic importance in diseases that are an outcome of splicing defects.

In another embodiment, the cargo is a DNA vector as described in co-owned and co-pending WO2022/251959, which is incorporated herein by reference. The DNA vector may be administered to a subject for the purpose of repairing, enhancing or blocking or reducing the expression of a cellular protein or peptide. Accordingly, the nucleotide polymers can be nucleotide sequences including genomic DNA, cDNA, or RNA.

As will be appreciated by those of skill in the art, the vector may encode promoter regions, operator regions or structural regions. The DNA vector may contain double-stranded DNA or may be composed of a DNA-RNA hybrid. Non-limiting examples of double-stranded DNA include structural genes, genes including operator control and termination regions, and self-replicating systems such as vector DNA.

Single-stranded nucleic acids include antisense oligonucleotides (complementary to DNA and RNA), ribozymes and triplex-forming oligonucleotides. In order to have prolonged activity, the single-stranded nucleic acids will preferably have some or all of the nucleotide linkages substituted with stable, non-phosphodiester linkages, including, for example, phosphorothioate, phosphorodithioate, phophoroselenate, or O-alkyl phosphotriester linkages.

The DNA vectors may include nucleic acids in which modifications have been made in one or more sugar moieties and/or in one or more of the pyrimidine or purine bases. Such sugar modifications may include replacement of one or more hydroxyl groups with halogens, alkyl groups, amines, azido groups or functionalized as ethers or esters. In another embodiment, the entire sugar may be replaced with sterically and electronically similar structures, including aza-sugars and carbocyclic sugar analogs. Modifications in the purine or pyrimidine base moiety include, for example, alkylated purines and pyrimidines, acylated purines or pyrimidines, or other heterocyclic substitutes known to those of skill in the art.

The DNA vector may be modified in certain embodiments with a modifier molecule such as a peptide, protein, steroid or sugar moiety. Modification of a DNA vector with such molecule may facilitate delivery to a target site of interest. In some embodiments, such modification translocates the DNA vector across a nucleus of a target cell. By way of example, a modifier may be able to bind to a specific part of the DNA vector (typically not encoding of the gene-of-interest), but also has a peptide or other modifier that has nucleus-homing effects, such as a nuclear localization signal. A non-limiting example of a modifier is a steroid-peptide nucleic acid conjugate as described by Rebuffat et al., 2002, Faseb J. 16(11):1426-8, which is incorporated herein by reference. The DNA vector may contain sequences encoding different proteins or peptides. Promoter, enhancer, stress or chemically-regulated promoters, antibiotic-sensitive or nutrient-sensitive regions, as well as therapeutic protein encoding sequences, may be included as required. Non-encoding sequences may be present as well in the DNA vector.

The nucleic acids used in the present disclosure can be isolated from natural sources, obtained from such sources as ATCC or GenBank libraries or prepared by synthetic methods. Synthetic nucleic acids can be prepared by a variety of solution or solid phase methods. Generally, solid phase synthesis is preferred. Detailed descriptions of the procedures for solid phase synthesis of nucleic acids by phosphite-triester, phosphotriester, and H-phosphonate chemistries are widely available.

In one embodiment, the DNA vector is double stranded DNA and comprises more than 700 base pairs, more than 800 base pairs or more than 900 base pairs or more than 1000 base pairs.

In another embodiment, the DNA vector is a nanoplasmid or a minicircle.

Gene editing systems can also be incorporated into delivery vehicles comprising the charged lipid. This includes a Cas9-CRISPR, TALEN and zinc finger nuclease gene editing system. In the case of Cas9-CRISPR, a guide RNA (gRNA), together with a plasmid or mRNA encoding the Cas9 protein may be incorporated into a delivery vehicle comprising the MC3-type lipid described herein. Optionally, a ribonucleoprotein complex may be incorporated into a delivery vehicle comprising the lipid described herein.

Likewise, the disclosure includes embodiments in which genetic material encoding DNA binding and cleavage domains of a zinc finger nuclease or TALEN system are incorporated into a delivery vehicle together with the MC3-type lipid of the disclosure.

While a variety of nucleic acid cargo molecules are described above, it will be understood that the above examples are non-limiting and the disclosure is not to be considered limiting with respect to the particular cargo molecule encapsulated in the delivery vehicle.

For example, the ionizable lipid described herein may also facilitate the incorporation of proteins and peptides into a delivery vehicle, which includes ribonucleoproteins. This includes both linear and non-linear peptides, proteins or ribonucleoproteins.

While pharmaceutical compositions are described above, the ionizable lipid described herein can be a component of any nutritional, cosmetic, cleaning or foodstuff product.

### Pharmaceutical Formulations

In some embodiments, the delivery vehicle comprising the cargo molecule is part of a pharmaceutical composition and is administered to treat and/or prevent a disease condition. The treatment may provide a prophylactic (preventive), ameliorative or a therapeutic benefit. The pharmaceutical composition will be administered at any suitable dosage.

In one embodiment, the pharmaceutical compositions is administered parentally, i.e., intra-arterially, intravenously, subcutaneously or intramuscularly. In yet a further embodiment, the pharmaceutical compositions are for intra-tumoral or in-utero administration. In another embodiment, the pharmaceutical compositions are administered intranasally, intravitreally, subretinally, intrathecally or via other local routes.

The pharmaceutical composition comprises pharmaceutically acceptable salts and/or excipients.

The compositions described herein may be administered to a patient. The term patient as used herein includes a human or a non-human subject.

The following examples are given for the purpose of illustration only and not by way of limitation on the scope of the invention.

### EXAMPLES

### Materials

The lipid 1,2-distearoyl-sn-glycero-3-phosphorylcholine (DSPC) and 1,2-dimyristoyl-rac-glycero-3-methoxypolyethylene glycol-2000 (PEG-DMG) were purchased from Avanti Polar Lipids™ (Alabaster, AL). Cholesterol and 10× Phosphate Buffered Saline (pH 7.4) were purchased from Sigma Aldrich™ (St Louis, MO). The ionizable amino-lipid was synthesized as previously described in WO 2022/246555, which is incorporated herein by reference.

An mRNA encoding firefly luciferase purchased from APExBIO Technology LLC (Houston, TX) was used to analyse luciferase activity.

### Methods

Preparation of Lipid Nanoparticles (LNP) Containing mRNA or siRNA

Lipids used in the formulation, nor-MC3, 4, DSPC, cholesterol, and PEG-DMG, were dissolved in ethanol at the appropriate ratios to a final concentration of 10 mM total lipid. Nucleic acid (mRNA) was dissolved in an appropriate buffer such as 25 mM sodium acetate pH 4 or sodium citrate pH 4 to a concentration necessary to achieve the appropriate amine-to-phosphate ratios. The aqueous and organic solutions were mixed using a rapid-mixing device as described in Kulkami et al., 2018, ACS Nano, 12:4787 and Kulkami et al., 2017, Nanoscale, 36:133347 (each incorporated herein by reference) at a flow rate ratio of 3:1 (v/v; respectively) and a total flow rate of 20 mL/min. The resultant mixture was dialyzed directly against 1000-fold volume of PBS pH 7.4. All formulations were concentrated using an Amicon™ centrifugal filter unit and analysed using the methods described below.

### Analysis of LNP

Particle size analysis of LNPs in PBS was carried out using backscatter measurements of dynamic light scattering with a Malvern Zetasizer™ (Worcestershire, UK). The reported particle sizes correspond to the number-weighted average diameters (nm). Total lipid concentrations were determined by extrapolation from the cholesterol content, which was measured using the Cholesterol E-Total Cholesterol Assay (Wako Diagnostics, Richmond, VA) as per the manufacturer's recommendations. Encapsulation efficiency of the formulations was determined using the Quant-iT RiboGreen™ Assay kit (Invitrogen, Waltham, MA). Briefly, the total mRNA content in solution was measured by lysing lipid nanoparticles in a solution of Tris-EDTA (TE) containing 2% Triton Tx-100, and free DNA vector in solution (external to LNP) was measured based on the RiboGreen™ fluorescence in a TE solution without Triton. Total mRNA content in the formulation was determined using a modified Bligh-Dyer extraction procedure. Briefly, LNP formulations containing mRNA were dissolved in a mixture of chloroform, methanol, and PBS that results in a single phase and the absorbance at 260 nm measured using a spectrophotometer.

### In Vivo Analysis in CD-1 Mice

LNP-mRNA encoding firefly luciferase were injected intravenously (tail-vein) into 6-8 week old CD-1 mice. Four hours following injection, the animals were euthanized and the liver and spleen were isolated. Tissue was homogenized in Glo Lysis™ buffer and a luciferase assay performed using the Steady Glo Luciferase™ assay kit (as per manufacturers recommendations).

### Organic Synthesis of Lipids 4-17

Unless otherwise specified, all reagents and solvents were commercial products and were used without further purification, except THF (freshly distilled from Na/benzophenone under Ar) and CH₂Cl₂ (freshly distilled from CaH₂ under Ar). “Dry methanol” was freshly distilled from magnesium turnings. All reactions were performed under an argon atmosphere. Reaction mixture from aqueous workups were dried by passing over a plug of anhydrous Na₂SO₄ held in a filter tube and rotary-evaporated under reduced pressure. Thin-layer chromatography was performed on silica gel plates coated with silica gel (Merck 60 F254 plates) and column chromatography was performed on 230-400 mesh silica gel. Visualization of the developed chromatogram was performed by staining with I₂ or potassium permanganate solution. Nuclear magnetic resonance spectra, ¹H (400 MHz) and ¹³C NMR (100 MHz), were recorded at room temperature in CDCl₃ solutions. ¹H NMR spectra were referenced to residual CHCl₃ (7.26 ppm) and ¹³C NMR spectra were referenced to the central line of the CDCl₃ triplet (77.00 ppm). Chemical shifts are reported in parts per million (ppm) on the 6 scale. Multiplicities are reported as “s” (singlet), “d” (doublet), “t” (triplet), “q” (quartet), “m” (multiplet), and further qualified as “app” (apparent) and “br” (broad). Low- and high-resolution mass spectra (m/z) were obtained in the electrospray (ESI) and field desorption/field ionisation (FD/FI) mode.

### Example 1: Lipid Synthesis

(i) Monomethyl azelate, 38. A solution of dimethyl azelate (50.0 g, 231.5 mmol, 1 equiv) and Ba(OH)₂ (43.8 g, 138.6 mmol, 1.2 equiv OH) in methanol (300 mL) was stirred at room

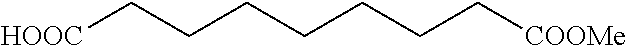 [SMILES: COC(=O)CCCCCCCC(=O)O]

temperature overnight. A heavy white precipitate formed. The solid was collected by vacuum filtration and washed with fresh MeOH (3×30 mL). The solid was suspended in 4N aq. HCl solution (200 mL) and the resulting mixture was extracted with CH₂Cl₂ (4×60 mL). The combined extracts were washed with brine (2×50 mL), dried (Na₂SO₄) and evaporated to give 43.0 g of crude product (92%), which was used directly in the next step. ¹H NMR (300 MHz, CDCl₃): δ 11.60 (s, 1H) 3.55 (s, 3H), 2.21 (m, 4H), 1.52 (m, 4H), 1.22 (m, 6H). ¹³C NMR (75 MHz, CDCl₃): δ 179.6, 174.1, 51.2, 33.7 (2 peaks), 28.6 (3 peaks) 24.6, 24.3.

(ii) Methyl 9-hydroxynonanoate, 39. To a cold (0° C.) THF (100 mL) solution of 38 (12.5 g, 61.8 mmol), maintained under N₂, in was added BH₃-DMS (7.32 mL, 80.4 mmol). The

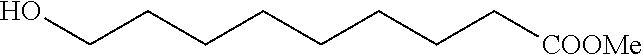 [SMILES: COC(=O)CCCCCCCCO]

mixture was warmed to room temperature and stirred for 90 minutes, at which point ¹H NMR confirmed consumption of starting material. The mixture was cooled to 0° C. and quenched carefully with H₂O (20.0 mL). The mixture was then extracted with DCM (3×40.0 mL). The combined organics were washed (brine), dried (Na₂SO₄), and concentrated to yield 39 (10.9 g, 94%). ¹H NMR: δ 3.64 (s, 3H), 3.60 (t, J=6.8 Hz, 2H), 2.27 (t, J=1.2 Hz, 2H), 1.62-1.47 (m, 4H), 1.28 (s, 8H). ¹³C NMR: a 174.2, 62.9, 51.4, 34.0, 32.7, 29.1 (2 peaks), 29.0, 25.6, 24.8. LRMS (ESI) 201 [M+Na]⁺.

(iii) Methyl 9-((methylsulfonyl)oxy)nonanoate, 40. To a solution of 39 (10.2 g, 54.1 mmol) and TEA (10.0 mL, 71.7 mmol) in DCM (70.0 mL) was added MsCl (5.03 mL, 65.0

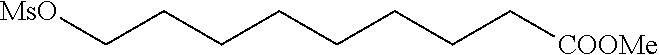 [SMILES: COC(=O)CCCCCCCCOS(C)(=O)=O]

mmol) at 0° C. under an atmosphere of nitrogen. The mixture was warmed to rt and stirred for 18 hours, at which point it was diluted with water (40.0 mL) and extracted with DCM (3×50.0 mL). The combined organics were washed (brine), dried (Na₂SO₄), and concentrated to yield crude 40 (14.2 g, ˜quant.), which was advanced to the next step without purification. ¹H NMR: δ 4.21 (t, J=6.5 Hz, 2H), 3.66 (s, 3H), 3.00 (s, 3H), 2.30 (t, J=7.5 Hz, 2H), 1.73 (dt, J=8.3, 6.5 Hz, 2H), 1.65-1.56 (m, 2H), 1.45-1.24 (m, 8H).

(iv) Methyl 9-(acetylthio)nonanoate, 41. To a slurry of K₂CO₃ (13.3 g, 96.3 mmol) and crude 40 (14.2 g, crude) in THF (100 mL), under N₂ atmosphere, was added thioacetic acid

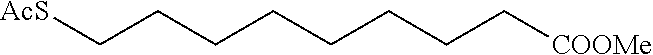

(6.11 mL, 86.8 mmol) at rt. The mixture was refluxed for 48 hours, cooled to room temperature and diluted with water (100 mL). The mixture was extracted with DCM (3×100 mL). The combined organics were washed (brine), dried (Na₂SO₄), and concentrated to yield crude 41 (13 g), which was advanced to the next step without purification. ¹H NMR (400 MHz, CDCl₃) δ 3.66 (s, 3H), 2.85 (t, J=7.4 Hz, 2H), 2.32 (s, 3H), 2.28 (t, J=7.7 Hz, 2H), 1.68-1.47 (m, 4H), 1.38-1.23 (m, 8H).

(v) (Chloromethyl)(pentyl)sulfane, 42. Gaseous HCl was bubbled through a cold (−15° C., ice/salt bath) solution of 1-pentanethiol (0.23 g, 2.2 mmol) in dry DCM (2 mL) containing

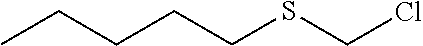

suspended paraformaldehyde (0.10 g, 3.3 mmol) and maintained under argon (balloon, needle vent). The mixture was stirred for 2 hours at −15° C. The solvent was removed under reduced pressure and DI H₂O was added to the residue until all the solid dissolved (ca. 5 mL). The mixture was extracted with Et₂O (3×5 mL) and the combined extracts were sequentially washed with saturated aqueous sodium bicarbonate solution (3×5 mL), water (3×5 mL), saturated aqueous sodium chloride solution (2×5 mL), dried over Na₂SO₄ and concentrated on a rotary evaporator to afford 0.34 g of 42 (quantitative) as a colorless oil. Compound 42 and related substances are sensitive materials that are best used without purification. ¹H NMR (300 MHz, CDCl₃) δ 4.77 (s, 2H), 2.76 (t, 2H), 1.68 (m, 2H), 1.39 (m, 4H), 0.93 (t, 3H) ppm. ¹³C NMR (75 MHz, CDCl₃) δ 49.9, 31.6, 30.9, 28.3, 22.2, 13.9.

(vi) Methyl 9-(((pentylthio)methyl)thio)nonanoate, 44. Thioacetate 41 (25.0 g, 101

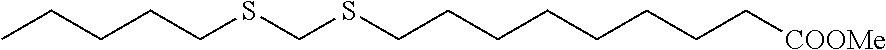 [SMILES: CCCCCSCSCCCCCCCCC(=O)OC]

mmol) was added via syringe to a degassed (N₂ bubbling for 20 min), well-stirred solution of NaOMe (5.5 g, 101 mmol) in DMF (500 mL) at room temperature. After stirring for 20 minutes, no more 41 was apparent by TLC. Chlorosulfane 42 (15.5 g, 101 mmol) was cautiously added via syringe to the well-stirred solution. The reaction was complete after 45 min, whereupon it was quenched with water and extracted with hexanes. The layers were separated and the organic phase was collected. The aqueous phase was back-extracted with hexanes (2×50 mL). The combined extracts were dried (Na₂SO₄), filtered and evaporated to yield the crude product (29.4 g, 90%) as a yellow oil. This material can be advanced to the following step, but if desired, it can be further purified by silica chromatography (1.5% EtOAc in hexanes). ¹H NMR. δ 3.66 (s, 3H), 3.65 (s, 2H), 2.62 (td, J=7.4, 1.4 Hz, 4H), 2.25 (t, J=7.5 Hz, 2H), 1.65-1.52 (m, 8H), 1.48-1.26 (m, 10H), 0.90 (t, J=7.1 Hz, 3H).

(vii) Methyl 9-((2-hydroxyethyl)thio) nonanoate, 45. Mesylate 40 (0.68 g, 1.0 mmol 1.0 g,

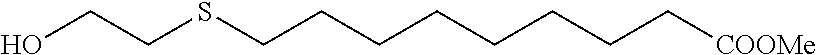 [SMILES: COC(=O)CCCCCCCCSCCO]

3.7 mmol) was added to a solution of 2-mercaptoethanol (0.47 g, 6.0 mmol), sodium borohydride (0.076 g, 2.0 mmol) and K₂CO₃ (0.97 g, 7.0 mmol) in anhydrous 60% MeOH/THF (5 mL), and the resulting mixture was stirred at room temperature for 14 h. The solvent was removed under reduced pressure and the residue was dissolved in ethyl acetate (5 mL) and sequentially washed with saturated aqueous ammonium chloride solution (2×5 mL), saturated aqueous sodium bicarbonate solution (2×5 mL), water (2×5 mL), and saturated aqueous sodium chloride solution (2×5 mL). The organic phase was dried over anhydrous sodium sulfate, filtered through Celite®, and concentrated under reduced pressure. The residue was purified by column chromatography on silica gel (20-40% EtOAc in hexanes) to afford 918 mg of product (70%) as a colorless oil. ¹H NMR: δ 3.70 (t, J=5.9 Hz, 2H), 3.65 (s, 3H), 2.71 (t, J=6.1 Hz, 2H), 2.50 (t, J=7.4 Hz, 2H), 2.29 (t, J=7.5 Hz, 2H), 2.13 (br, 1H), 1.71-1.45 (m, 4H), 1.44-1.15 (m, 8H). ¹³C NMR: δ 174.4, 60.3, 51.6, 35.4, 34.2, 31.7, 29.8, 29.2, 29.14, 29.1, 28.8, 25.0.

(viii) Methyl 9-((2-chloroethyl)thio)nonanoate (46). A solution of methanesulfonyl

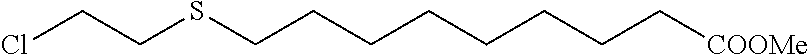 [SMILES: COC(=O)CCCCCCCCSCCCl]

chloride (0.17 g, 1.47 mmol) in anhydrous dichloromethane (1 mL) was added dropwise to a cold (0° C.) solution of 45 (0.45 g, 0.70 mmol 347 mg, 1.4 mmol) and triethylamine (0.17 g, 1.68 mmol) in 2 mL of anhydrous CH₂Cl₂, under argon. Upon completion of the addition, the reaction mixture was allowed to warm to ambient temperature and stirred overnight. The reaction mixture was diluted with CH₂Cl₂ (5 mL) and sequentially washed with saturated aqueous sodium bicarbonate solution (3×5 mL), saturated aqueous ammonium chloride solution (2×5 mL), water (3×5 mL), and saturated aqueous sodium chloride solution (3×5 mL). The organic phase was dried over anhydrous sodium sulfate, filtered through Celite®, and concentrated under reduced pressure to afford 360 mg of product as a pale yellow oil, which was used in the next step without purification. ¹H NMR: δ 3.66 (s, 3H), 3.61 (t, J=7.4 Hz, 2H), 2.84 (t, J=7.5 Hz, 2H), 2.54 (t, J=7.4 Hz, 2H), 2.29 (t, J=7.5 Hz, 2H), 1.69-1.50 (m, 4H), 1.45-1.24 (m, 8H). ¹³C NMR: δ 174.4, 51.6, 43.2, 34.4, 34.2, 32.6, 29.8, 29.2, 29.16, 29.1, 28.8, 25.0.

(ix) Methyl 9-((2-(butylthio)ethyl)thio)nonanoate, 47. Crude compound 46 (360 mg) was added to a solution of 1-butanethiol (340 mg, 3.7 mmol), sodium borohydride (47 mg, 1.2

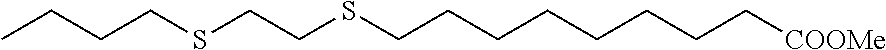 [SMILES: CCCCSCCSCCCCCCCCC(=O)OC]

mmol) and K₂CO₃ (600 mg, 4.34 mmol) in anhydrous 60% MeOH/THF (2 mL), and the resulting mixture was stirred at room temperature for 14 h. The solvent was removed under reduced pressure and the residue was dissolved in ethyl acetate (5 mL) and sequentially washed with saturated aqueous sodium bicarbonate solution (3×5 mL), water (3×5 mL), and saturated aqueous sodium chloride solution (2×5 mL). The organic phase was dried over anhydrous sodium sulfate, filtered through Celite®, and concentrated under reduced pressure. The residue was purified by column chromatography on silica gel (0-5% ethyl acetate in hexane) to afford 269 mg of 47 (60% over 2 steps) as a pale yellow oil. ¹H NMR: δ 3.61 (s, 3H), 2.66 (s, 4H), 2.52-2.46 (m, 4H), 2.25 (t, J=7.5 Hz, 2H), 1.67-1.45 (m, 4H), 1.45-1.19 (m, 12H), 0.87 (t, J=7.3, 3H). ¹³C NMR: δ 174.2, 51.4, 38.9, 34.1, 32.2, 31.9, 31.8, 31.3, 29.7, 29.13, 29.06, 29.0, 28.8, 24.9, 22.0, 13.7.

(x) Methyl 9-((3-hydroxypropyl)thio)nonanoate, 48. Prepared according to procedure vii above, except that 3-mercapto-1-propanol was used in lieu of 2-mercaptoethanol. Yield: 510

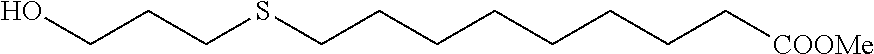 [SMILES: COC(=O)CCCCCCCCSCCCO]

mg (75%) as a colorless oil. ¹H NMR: δ 4.20 (t, J=6.5 Hz, 1H), 3.84-3.72 (m, 2H), 3.65 (s, 3H), 2.69-2.59 (m, 4H), 2.29 (t, J=7.5 Hz, 2H), 1.90-179 (m, 2H), 1.79-1.65 (m, 4H), 1.60-1.52 (m, 2H), 1.48-1.21 (m, 6H). ¹³C NMR: δ 174.5, 61.1, 51.6, 36.3, 34.2, 32.2, 32.0, 29.6, 29.2, 29.15, 29.1, 28.9, 25.0.

(xi) Methyl 9-((3-((methylsulfonyl)oxy)propyl)thio)nonanoate, 49. Prepared from 510

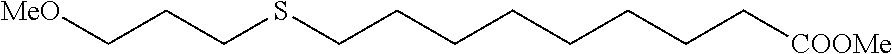 [SMILES: COCCCSCCCCCCCCC(=O)OC]

mg of compound 48 according to procedure viii above. Yield: 620 mg as a pale-yellow oil, which was used in the next step without further purification. ¹H NMR: δ 4.34 (t, J=6.2 Hz, 2H), 3.65 (s, 3H), 3.01 (s, 3H), 2.62 (t, J=7.0 Hz, 2H), 2.49 (t, J=7.5 Hz, 2H), 2.29 (t, J=7.5 Hz, 2H), 2.06-1.97 (m, 2H), 1.65-1.51 (m, 4H), 1.41-1.21 (m, 8H). ¹³C NMR: δ 174.4, 68.5, 51.6, 37.5, 34.2, 32.3, 29.6, 29.2, 29.17, 29.1, 29.06, 28.9, 27.9, 25.0.

(xii) Methyl 9-((3-(propylthio)propyl)thio)nonanoate, 50. Prepared from crude mesylate 49 (620 mg) according to procedure ix above, except that 1-propanethiol was used in lieu

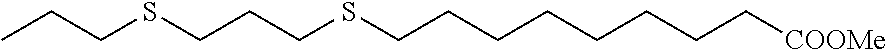 [SMILES: CCCSCCCSCCCCCCCCC(=O)OC]

of 1-butanethiol. Yield: 417 mg (67%) as a pale yellow oil. ¹H NMR: δ 3.62 (s, 3H), 2.56 (t, J=7.2 Hz, 4H), 2.45 (t, J=7.4 Hz, 4H), 2.25 (t, J=7.5 Hz, 2H), 1.91-1.74 (m, 2H), 1.63-1.47 (m, 6H), 1.38-1.16 (m, 8H), 0.94 (t, J=7.4 Hz, 3H). ¹³C NMR: δ 174.3, 51.4, 34.2, 34.1, 32.2, 31.0, 30.9, 29.6, 29.5, 29.15, 29.1, 29.0, 28.8, 24.9, 23.0, 13.5.

(xiii) Methyl 3-oxo-11-(((pentylthio)methyl)thio)-2-(7-(((pentylthio)methyl) thio)heptyl)undecanoate, 51. To a solution of 44 (4.24 g, 12.2 mmol) and tributylamine (5.20 mL, 21.9 mmol) in toluene (35.0 mL) was added dropwise a solution of TiCl₄ (2.00

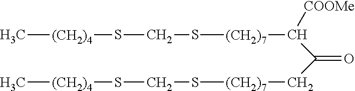

mL, 18.3 mmol) in toluene (10.0 mL) at 0° C. under nitrogen over the course of 15 minutes. After addition, the mixture was warmed to room temperature and stirred for 90 minutes. Water (30.0 mL) was added and the mixture was extracted with CH₂Cl₂ (3×40.0 mL). The combined extracts were washed (brine), dried (Na₂SO₄) and concentrated to give 51 as a mixture of keto- and enol forms. The compound was advanced to the next step without purification. ¹H NMR (CDCl₃, keto form) δ 3.66 (s, 3H), 3.65 (s, 4H), 3.09 (br dd, 1H) 2.62 (two t, J=7.4, 8H), 2.41 (t, J=7.5 Hz, 2H), 1.57 (m, 14H), 1.44-1.22 (m, 22H), 0.90 (t, J=7.1 Hz, 6H).

(xiv) Methyl 11-((2-(butylthio)ethyl)thio)-2-(7-((2-(butylthio)ethyl)thio)heptyl)-3-oxoundecanoate, 52. Prepared from ester 47 in 77% yield according to procedure xiii

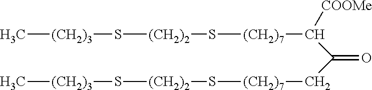

above. ¹H NMR (300 MHz, CDCl₃): δ 3.69 (s, 3H), 3.42 (t, J=7.3 Hz, 1H), 2.70 (s, 8H), 2.59-2.46 (m, 10H), 1.90-1.78 (m, 1H), 1.76-1.62 (m, 1H), 1.64-1.45 (m, 10H), 1.45-1.16 (m, 20H), 0.89 (t, J=7.2 Hz, 6H). ¹³C NMR (75 MHz, CDCl₃): δ 205.4, 170.5, 52.4, 52.2, 41.9, 38.9, 34.1, 32.8, 32.2, 32.0, 31.8, 31.4, 29.75, 29.7, 29.3, 29.28, 29.2, 29.1, 29.07, 29.0, 28.9, 28.8, 28.3, 27.5, 25.8, 25.2, 22.0, 20.3, 13.6.

(xv) Methyl 3-oxo-11-((3-(propylthio)propyl)thio)-2-(7-((3-(propylthio)propyl)thio) heptyl)undecanoate, 53. Prepared in 74% yield from ester 50 according to procedure xiii above. ¹H NMR: δ 3.69 (s, 3H), 3.40 (t, J=7.5 Hz, 1H), 2.58 (t, J=7.5 Hz, 8H), 2.49-2.44

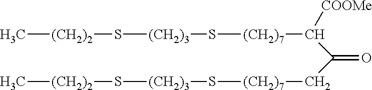

(m, 10H), 1.87-1.70 (m, 4H), 1.64-1.52 (m, 12H), 1.38-1.16 (m, 16H), 0.95 (t, J=7.2 Hz, 6H). ¹³C NMR: δ 205.5, 170.5, 52.2, 51.6, 42.0, 34.3, 32.2, 32.19, 31.1, 31.0, 29.7, 29.65, 29.5, 29.3, 29.31, 29.2, 29.1, 29.11, 29.0, 28.99, 28.9, 28.8, 28.3, 27.5, 25.2, 23.0, 20.3, 13.6.

(xvi) 6,8,26,28-Tetrathiatritriacontan-17-one, 54. Crude 51 obtained as described in part (xiii) above was dissolved in EtOH (20 mL) and 4 N NaOH (5 mL) was added. The mixture

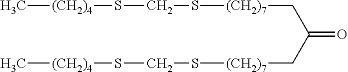

was stirred at 60° C. for 18 hours, cooled to room temperature and acidified to pH 1 with conc. HCl. The mixture was concentrated to roughly 40% volume and extracted with CH₂Cl₂ (3×30.0 mL). The combined extracts were washed (brine), dried (Na₂SO₄) and concentrated. The residue was purified by silica chromatography (1.5% EtOAc in Hexanes) to yield 51 (2.3 g, 67% from 44). ¹H NMR (CDCl₃) δ 3.65 (s, 4H), 2.62 (t, J=7.4 Hz, 4H), 2.61 (t, J=7.4 Hz, 4H), 2.38 (t, J=7.5 Hz, 4H), 1.57 (m, 14H), 1.44-1.22 (m, 22H), 0.90 (t, J=7.1 Hz, 6H).

(xvii) 5,8,26,29-Tetrathiatritriacontan-17-one, 55. Prepared in 86% yield from ketoester

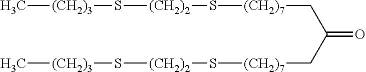

52 according to procedure xvi above. ¹H NMR (300 MHz, CDCl₃): δ 2.69 (s, 8H), 2.55-249 (m, 12H), 1.64-1.47 (m, 12H), 1.44-1.18 (m, 20H), 0.89 (t, J=7.3 Hz, 6H).

(xviii) 4,8,26,30-Tetrathiatritriacontan-17-one, 56. Prepared in 89% yield from ketoester 53 according to procedure xvi above. ¹H NMR: δ 2.59 (t, J=7.2 Hz, 8H), 2.48 (t, J=7.5

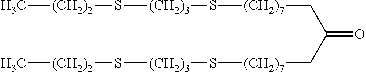

### Hz, 8H), 2.36 (t, J=7.4 Hz, 4H), 1.84 (p, J=7.2 Hz, 4H), 1.65-1.43 (m, 12H), 1.43-1.12 (m, 16H), 0.95 (t, J=7.3 Hz, 6H). ¹³C NMR: δ 211.6, 42.8, 34.2, 32.2, 31.0, 29.6, 29.5, 29.3, 29.2, 29.1, 28.8, 25.3, 23.8, 23.0, 13.5.

(xix) 2-(2,2-bis(8-(((Pentylthio)methyl)thio)octyl)-1,3-dioxolan-4-yl)ethan-1-ol, 57. A

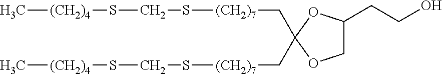

mixture of ketone 54 (100 mg, 0.181 mmol), 1,2,4-butanetriol (38.5 mg, 0.363 mmol) and pyridinium p-toluenesulfonate (9.09 mg, 0.0362 mmol) in 4.00 mL of toluene was refluxed under nitrogen overnight with continuous removal of water (Dean-Stark trap). Upon completion of the reaction, the mixture was cooled to room temperature, washed with water (2×5.00 mL), brine (5.00 mL) then dried (Na₂SO₄) and concentrated. The residue was purified by silica gel column chromatography (0-3% MeOH in DCM) to yield ketal 57 (91.6 mg, 79%). ¹H NMR (400 MHz, CDCl₃) δ 4.26-4.18 (m, 1H), 4.07 (dd, J=7.9, 6.0 Hz, 1H), 3.82-3.76 (m, 2H), 3.64 (s, 4H), 3.52 (t, J=8.0 Hz, 1H), 2.61 (t, J=7.5 Hz, 8H), 1.86-1.75 (m, 2H), 1.66-1.49 (m, 14H), 1.45-1.21 (m, 26H), 0.95-0.82 (m, 6H).

(xx) 2-(2,2-bis(8-((2-(Butylthio)ethyl)thio)octyl)-1,3-dioxolan-4-yl)ethan-1-ol, 58.

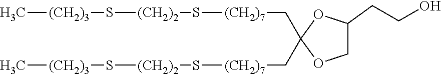

Prepared from ketone 55 according to procedure xix above. ¹H NMR: δ 4.23 (dd, J=7.8, 5.9 Hz, 1H), 4.08 (dd, J=7.9, 6.0 Hz, 1H), 3.80 (t, J=5.7 Hz, 2H), 3.52 (t, J=8.0 Hz, 1H), 2.71 (s, 8H), 2.54 (t, J=7.4, 8H), 1.83-1.77 (m, 2H), 1.67-1.47 (m, 13H), 1.47-1.20 (m, 24H), 0.91 (t, J=7.3 Hz, 6H). ¹³C NMR (75 MHz, CDCl₃) δ 112.7, 75.7, 70.1, 61.0, 42.9, 37.9, 37.4, 35.5, 34.8, 32.3, 32.0, 30.0, 29.95, 29.8, 29.7, 29.3, 29.2, 29.0, 24.1, 23.9, 22.1, 13.8.

(xxi) 2-(2,2-bis(8-((3-(Propylthio)propyl)thio)octyl)-1,3-dioxolan-4-yl)ethan-1-ol, 59.

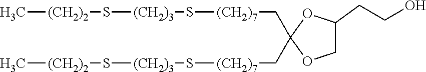

Prepared from ketone 56 according to procedure xix above. ¹H NMR (CDCl₃) δ 4.27-4.15 (m, 1H), 4.08 (dd, J=7.9, 6.0 Hz, 1H), 3.79 (t, J=5.6 Hz, 2H), 3.54-3.49 (m, 1H), 2.61 (t, J=6.6 Hz, 8H), 2.51-2.41 (m, 8H), 1.97 (br, 1H), 1.89-1.72 (m, 4H), 1.86-1.52 (m, 12H), 1.45-1.16 (m, 22H), 0.98 (t, J=7.3 Hz, 6H). ¹³C NMR (CDCl₃) δ 112.7, 75.6, 70.1, 61.0, 42.9, 37.9, 37.4, 35.5, 34.3, 32.3, 32.2, 31.1, 30.0, 29.8, 29.8, 29.8, 29.6, 29.3, 29.2, 29.0, 29.0, 23.1, 13.7, 13.6.
(xxii) 2-(2,2-bis(8-(((Pentylthio)methyl)thio)octyl)-1,3-dioxolan-4-yl)ethyl methanesulfonate, 60. To a solution of ketal 57 (91.6 mg, 0143 mmol) and TEA (0.0298 mL, 0.215

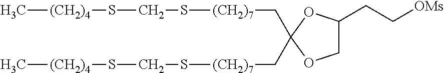

mmol) in DCM (2.00 mL) was added MsCl (0.0133 mL, 0.172 mmol) at a C under an atmosphere of nitrogen. The mixture was warmed to room temperature and stirred for 18 hours. The mixture was diluted with water (3.00 mL) and extracted with DCM (3×3.00 mL). The combined organics were washed (brine), dried (Na₂SO₄) and concentrated to yield crude mesylate 60 (105 mg). ¹H NMR (CDCl₃) δ 4.42-4.26 (m, 2H), 4.22-4.13 (m, 1H), 4.08 (dd, J=7.9, 6.1 Hz, 1H), 3.67 (s, 4H), 3.51 (t, J=7.7 Hz, 1H), 3.13 (s, 3H), 2.61 (t, J=7.5 Hz, 8H), 2.04-1.88 (m, 2H), 1.67-1.49 (m, 14H), 1.44-1.19 (m, 26H), 0.88 (t, J=7.1 Hz, 6H).

(xxiii) 2-(2,2-bis(8-((2-(butylthio)ethyl)thio)octyl)-1,3-dioxolan-4-yl)ethyl methanesulfonate, 61. Prepared from 58 according to procedure xxii above. ¹H NMR (CDCl₃) δ

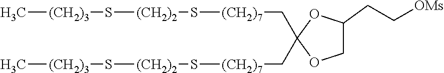

4.42-4.29 (m, 1H), 4.22-4.13 (m, 1H), 4.25-4.03 (m, 2H), 3.56-3.45 (m, 1H), 3.01 (s, 3H), 2.70 (s, 8H), 2.53 (t, J=7.4, 8H), 2.03-1.88 (m, 2H), 1.73-1.13 (m, 36H), 0.90 (t, J=7.3 Hz, 6H). ¹³C NMR (CDCl₃) δ 112.7, 72.4, 69.7, 67.2, 37.8, 37.4, 333.4, 32.3, 32.3, 32.0, 31.9, 29.9, 29.8, 29.6, 29.2, 28.9, 24.1, 23.9, 22.1, 13.8

(xxiv) 2-(2,2-bis(8-((3-(propylthio)propyl)thio)octyl)-1,3-dioxolan-4-yl)ethyl methanesulfonate, 62. Prepared from 59 according to procedure xxii above. ¹H NMR (CDCl₃) δ

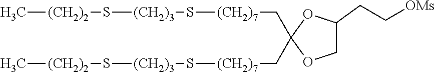

### 4.40-4.23 (m, 2H), 4.2-4.1 (m, 1H), 4.09-4.02 (m, 1H), 3.53-3.47 (m, 1H), 3.00 (s, 3H), 2.58 (t, J=6.9 Hz, 8H), 2.47-2.43 (m, 8H), 1.90-1.74 (m, 4H), 1.67-1.42 (m, 12H), 1.42-1.13 (m, 22H), 0.96 (t, J=7.3 Hz, 6H).

(xxv) 2-(2,2-bis(8-(((pentylthio)methyl)thio)octyl)-1,3-dioxolan-4-yl)-N,N-dimethyl-ethan-1-amine, 4. A solution of crude mesylate 60 (105 mg) in 2 N dimethylamine in THF

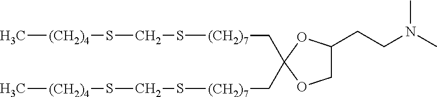

(5 mL) was stirred for 5 days at room temperature in a sealed flask. The mixture was then concentrated, and the residue was purified by silica gel chromatography (0-5% MeOH in CH₂Cl₂) to yield 21 (61.1 mg, 64% over 2 steps). ¹H NMR (C₆D₆) δ 4.10 (t, J=6.6 Hz, 1H), 3.95 (dd, J=7.8, 5.9 Hz, 1H), 3.50 (s, 4H), 3.44 (t, J=7.8 Hz, 1H), 2.61-2.49 (m, 8H), 2.37-2.19 (m, 2H), 2.02 (s, 6H), 1.87-1.74 (m, 4H), 1.68-1.44 (m, 12H), 1.42-1.11 (m, 26H), 0.83 (t, J=7.1 Hz, 6H). LRMS (ESI+) m/z 666 [M+H]⁺.

(xxvi) 2-(2,2-bis(8-((2-(butylthio)ethyl)thio)octyl)-1,3-dioxolan-4-yl)-N,N-dimethyl-ethan-1-amine, 5. Prepared from crude mesylate 61 according to procedure xxv above. ¹H

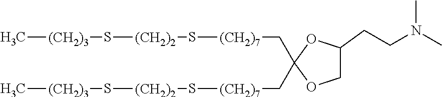

NMR (CDCl₃) δ 4.16-3.99 (m, 2H), 3.60-3.40 (m, 1H), 2.71 (s, 8H), 2.53 (t, J=7.2, 8H), 2.47-2.30 (m, 2H), 2.26 (s, 6H), 1.91-1.47 (m, 14H), 1.47-1.18 (m, 24H), 0.91 (t, J=7.1 Hz, 6H). ¹³C NMR (75 MHz, CDCl₃) δ 112.2, 74.8, 70.1, 56.4, 45.5, 37.9, 37.6, 32.4, 32.3, 31.9, 31.8, 30.02, 30.0, 29.6, 29.6, 29.4, 29.3, 29.0, 23.8, 22.1, 13.8. LRMS m/z 666 [M+H]⁺.

(xxvii) 2-(2,2-bis(8-((3-(propylthio)propyl)thio)octyl)-1,3-dioxolan-4-yl)-N,N-dimethylethan-1-amine, 6. Prepared from crude mesylate 62 according to procedure xxv

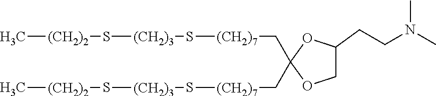

above. ¹H NMR (300 MHz, CDCl₃) δ 4.17-4.10 (m, 2H), 3.59-3.49 (m, 1H), 3.21-3.09 (m, 2H), 2.86-2.77 (m, 6H), 2.60 (t, J=7.2 Hz, 8H), 2.52-2.46 (m, 8H), 1.85 (p, J=7.2 Hz, 4H), 1.67-1.48 (m, 12H), 1.41-1.18 (m, 22H), 0.98 (t, J=7.3 Hz, 6H). ¹³C NMR (75 MHz, CDCl₃) δ 113.2, 73.2, 69.5, 44.2, 42.5, 37.7, 37.0, 34.4, 32.3, 31.2, 31.1, 29.8, 29.7, 29.6, 29.3, 29.0, 28.9, 24.1, 23.1, 13.7. LRMS m/z 666 [M+H]⁺.

(xxviii) 4-((2-(2,2-bis(8-(((Pentylthio)methyl)thio)octyl)-1,3-dioxolan-4-yl)ethyl)-(methyl)-amino)butan-1-ol, 10. Prepared from crude mesylate 60 (100 mg, 141 uM) and 4-(methylamino)-1-butanol according to procedure xxv above. ¹H NMR (C₆D₆): δ 4.10 (t, J=6.6 Hz,

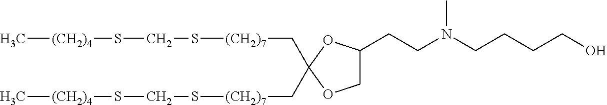

1H), 3.95 (dd, J=7.8, 5.9 Hz, 1H), 3.50 (s, 4H), 3.48 (t, J=7.0 Hz, 2H), 3.44 (t, J=7.8 Hz, 1H), 2.61-2.49 (m, 10H), 2.37-2.19 (m, 2H), 2.15 (s, 3H), 1.87-1.74 (m, 4H), 1.68-1.44 (m, 14H), 1.42-1.11 (m, 28H), 0.83 (t, J=7.1 Hz, 6H). LRMS (ESI+) m/z 724 [M+H]⁺.

(xxix) 2-(2,2-bis(8-((2-(Butylthio)ethyl)thio)octyl)-1,3-dioxolan-4-yl)ethyl 4-(dimethylamino)butanoate, 11. A solution of ketal 57 (166 mg, 260 umol, 1.0 equiv), 4-dimethylaminobutyric acid hydrochloride (312 umol, 52 mg, 1.2 equiv), and DMAP (6 mg,

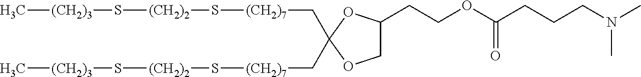

50 umol, 0.2 equiv) in dry CH₂Cl₂ (5 mL) was stirred at room temperature for 5 minutes prior to the addition of EDCI (390 umol, 75 mg, 1.5 equiv). The reaction was complete after overnight stirring. The mixture was quenched with water and extracted with CH₂Cl₂ (3×5 mL). The combined extracts were dried (Na₂SO₄) and evaporated, and the yellowish oily residue was purified by column chromatography on silica gel (40 mL) by eluting with 4-6% MeOH in CH₂Cl₂ to afford pure 23 (133 mg, 68%) as a colorless oil. ¹H NMR: δ 4.26-4.18 (m, 1H), 4.15-4.04 (m, 3H), 3.52 (t, J=8.0 Hz, 1H), 2.70 (s, 8H), 2.61 (t, J=7.5 Hz, 8H), 2.37-2.26 (m, 10H) 1.92-1.80 (m, 2H), 1.66-1.49 (m, 14H), 1.45-1.21 (m, 24H), 0.95-0.82 (m, 6H). LRMS m/z 752 [M+H]⁺.

(xxx) 3-((tert-Butyldiphenylsilyl)oxy)-N-methylpropan-1-amine. A solution of tert-butyl(chloro)-diphenylsilane (10.2 g, 37.0 mmol, 1.1 equiv) in CH₂Cl₂ (6 mL) was added

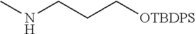

dropwise during 15 min to a well-stirred solution of 3-(methylamino)-1-propanol (3.0 g, 33.6 mmol, 1.0 equiv) and imidazole (5.1 g, 73.9 mmol, 2.2 equiv) in DCM (8 mL). The mixture was stirred overnight at room temperature. The reaction mixture was sequentially washed with sat. aq. NaHCO₃ solution (2×10 mL), water (2×10 mL), and sat. aq. NaCl chloride solution (2×10 mL), then dried over anhydrous Na₂SO₄, filtered, and concentrated under reduced pressure to furnish 52 (11.0 g, 91%) as a yellow oil. ¹H NMR: δ 7.71-7.68 (m, 4H), 7.40-7.36 (m, 6H), 3.69 (t, J=6.0 Hz, 2H), 2.67 (t, J=6.6 Hz, 2H), 2.66 (s, 3H), 1.65-1.48 (m, 2H), 1.09 (s, 9H). ¹³C NMR δ 135.4, 133.8, 129.4, 127.5, 61.5, 47.5, 36.2, 33.2, 32.0, 26.7. LRMS m/z 328 [M+H]⁺.

### (xxxi) Ethyl 4-((3-((tert-butyldiphenylsilyl)oxy)propyl)(methyl)amino)butanoate

A solution of 3-((tert-butyldiphenylsilyl)oxy)-N-methylpropan-1-amine (330 mg, 1.0

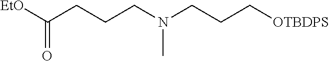

mmol) and ethyl 4-bromobutyrate (250 mg, 1.3 mmol) in MeOH (8 mL) in a sealed microwave reaction vial was microwave irradiated for 15 minutes (normal absorption, 115° C.), then it was cooled to room temperature and concentrated. The residue was purified by silica gel column chromatography (0-20% MeOH in DCM) to yield the desired 53 (309 mg, 70%). ¹H NMR: δ 7.71-7.68 (m, 4H), 7.40-7.36 (m, 6H), 4.1 (q, J=6.6 Hz, 2H), 3.72 (t, J=6.2 Hz, 2H), 2.68 (m, 4H), 2.60 (s, 3H), 2.47 (t, J=6.8 Hz, 2H), 1.65-1.48 (m, 4H), 1.09 (s, 9H); 1.07 (t, J=6.6 Hz, 3H). LRMS m/z 442 [M+H]⁺.

(xxxii) 4-((3-((tert-Butyldiphenylsilyl)oxy)propyl)(methyl)amino)butanoic acid hydrochloride. A solution of ethyl 4-((3-((tert-butyldiphenylsilyl)oxy)propyl)(methyl)amino)butanoate (300 mg, 680 umol) and aqueous 1 N LiOH (1 ml, 1 mmol) in THF (2 mL)

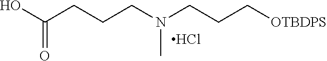

was stirred at room temperature overnight, whereupon saponification was complete. The solution was cooled to 0° C. and acidified to pH 4 with 1 N HCl, then it was concentrated. The aqueous residue was extracted with ethyl acetate (3×2 mL) and the combined extracts were filtered over a plug of Na₂SO₄ and concentrated to dryness. The crude acid thus obtained appeared to contain some inorganic matter, but it was used directly in the next step without purification. LRMS m/z 414 [M+H]⁺.

(xxxiii) 2-(2,2-bis(8-((2-(Butylthio)ethyl)thio)octyl)-1,3-dioxolan-4-yl)ethyl 4-((3-((tert-butyldiphenylsilyl)oxy)propyl)(methyl)amino)butanoate, 63. Prepared in 71% yield

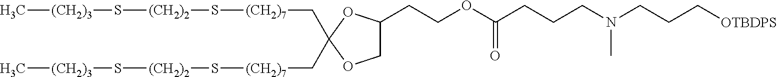

from ketal 57 and crude 4-((3-((tert-butyldiphenylsilyl)oxy)propyl)(methyl)amino) butanoic acid hydrochloride according to procedure xxix above. ¹H NMR: δ 7.71-7.68 (m, 4H), 7.40-7.36 (m, 6H), 4.26-4.18 (m, 1H), 4.15-4.04 (m, 3H), 3.72 (t, J=6.2 Hz, 2H), 3.52 (t, J=7.0 Hz, 1H), 2.70 (s, 8H), 2.61 (t, J=7.5 Hz, 8H), 2.68 (m, 4H), 2.60 (s, 3H), 2.47-2.26 (m, 10H), 1.92-1.49 (m, 12H), 1.45-1.21 (m, 22H), 1.09 (s, 9H), 0.90 (br t, 6H). LRMS m/z 1034 [M+H]⁺.

(xxxiv) 2-(2,2-bis(8-((2-(Butylthio)ethyl)thio)octyl)-1,3-dioxolan-4-yl)ethyl 4-((3-hydroxypropyl)(methyl)amino)butanoate, 12. Pyridine-HF complex (70% w/w, 23 uL,

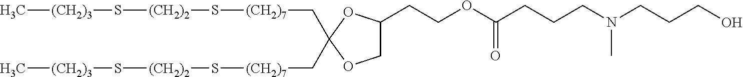

4 equiv) and pyridine (30 uL) were added to a cold (0° C.) solution of 63 (210 mg, 203 umol, 1 equiv) in 0.5 mL of anhydrous THF. The mixture was allowed to warm to ambient temperature and stirred until TLC showed complete desilylation. Saturated aq. NaHCO₃ solution (0.5 mL) and tert-butyl methyl ether (0.5 mL; tBuOMe reduces the extent of formation of a gelatinous precipitate and greatly facilitates the extraction of the product) were added to the reaction mixture at 0° C. The aqueous phase was extracted with diethyl ether (2×5 mL), the combined extracts were dried over Na₂SO₄, filtered and evaporated, and the residue was purified by flash chromatography (2% MeOH/DCM) to give 12 as a colorless oil (125 mg, 77%). ¹H NMR: δ 4.26-4.18 (m, 1H), 4.10 (m, 1H), 3.68 (t, J=6.2 Hz, 2H), 3.52 (t, J=7.0 Hz, 1H), 2.70 (s, 8H), 2.61 (t, J=7.5 Hz, 8H), 2.68 (m, 4H), 2.60 (s, 3H), 2.47-2.26 (m, 10H), 1.92-1.49 (m, 12H), 1.45-1.21 (m, 24H), 0.90 (br t, 6H). LRMS m/z 796 [M+H]⁺.

(xxxv) 4-((tert-Butyldiphenylsilyl)oxy)butan-1-amine. A solution of tert-butyl(chloro)-

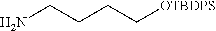

diphenylsilane (6.8 g, 24.7 mmol, 1.1 equiv) in CH₂Cl₂ (4 mL) was added dropwise during 15 min to a well-stirred solution of 4-amino-1-butanol (2.0 g, 22.4 mmol, 1.0 equiv) and imidazole (3.4 g, 49.3 mmol, 2.2 equiv) in DCM (5 mL). The mixture was stirred overnight at room temperature. The reaction mixture was sequentially washed with sat. aq. NaHCO₃ solution (2×5 mL), water (2×5 mL), and sat. aq. NaCl chloride solution (2×5 mL), then dried over anhydrous Na₂SO₄, filtered, and concentrated under reduced pressure to give the product (6.72 g, 92%) as a yellow oil. ¹H NMR (300 MHz, CDCl₃): δ 7.71-7.68 (m, 4H), 7.40-7.36 (m, 6H), 3.70 (t, J=6.0 Hz, 2H), 2.67 (t, J=6.6 Hz, 2H), 1.86 (s, 2H), 1.65-1.48 (m, 4H), 1.09 (s, 9H); ¹³C NMR (75 MHz, CDCl₃): δ 135.4, 133.8, 129.4, 127.5, 63.6, 41.8, 29.9, 29.8, 26.7, 19.0; LRMS: 328 [M+H]⁺.

(xxxvi) N-(4-((tert-butyldiphenylsilyl)oxy)butyl)-6,8,26,28-tetrathiatritriacontan-17-amine, 64. Sodium triacetoxyborohydride (445 mg, 2.1 mmol, 1.5 equiv) was added at room

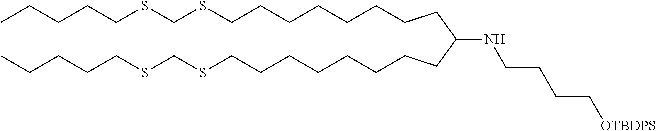

temperature and under argon to a vigorously stirred solution of ketone 54 (770 mg, 1.4 mmol, 1 equiv), 4-((tert-butyldiphenylsilyl)oxy)butan-1-amine (690 mg, 2.1 mmol, 1.5 equiv), and glacial acetic acid (1 drop) in (CH₂Cl)₂ (3 mL). The mixture was stirred at room temperature overnight, then it was diluted with more CH₂Cl₂ (5 mL), and treated with aq. sat. NaHCO₃ (2 mL). The organic phase was separated, sequentially washed with more aq. sat. NaHCO₃ (2 mL) and water (2×5 mL), dried (Na₂SO₄), and evaporated. The oily residue was purified by silica gel column chromatography (1% MeOH in CH₂Cl₂) to furnish 854 mg (70%) of 75 as a colorless oil. ¹H NMR: δ 7.71-7.68 (m, 4H), 7.40-7.36 (m, 6H), 3.65 (t, J=6.0 Hz, 2H), 3.50 (s, 4H), 2.60-2.40 (cm, 11H), 1.60-1.25 (cm, 44H), 1.09 (s, 9H), 0.90 (t, 6H). LRMS m/z 862 [M+H]⁺.

(xxxvii) N-(4-((tert-butyldiphenylsilyl)oxy)butyl)-N-methyl-6,8,26,28-tetrathiatritria-contan-17-amine, 65. A solution of 64 (200 mg, 232 umol) and aqueous 37% HCHO (500

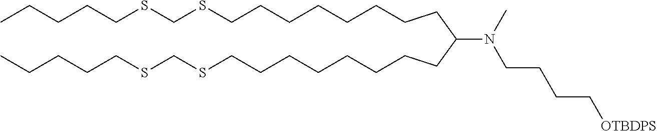

uL) in THF (4 mL) was stirred at room temperature and under argon prior to the addition of sodium triacetoxyborohydride (250 mg, 1.2 mmol, 5 equiv). After stirring at room temperature under Ar overnight, TLC showed the reaction to be complete. The solution was evaporated to dryness and the residue was partitioned between aqueous saturated NaHCO₃ solution (10 mL) and CH₂Cl₂ (10 mL). The organic layer was recovered and the aqueous phase was extracted twice more with CH₂Cl₂ (5 mL each time). The combined organic phases were dried (Na₂SO₄) and concentrated in vacuo, and the residue was purified by silica gel chromatography (0-44% MeOH in CH₂Cl₂) to give compound 65 (173 mg, 85%) as a colorless oil. ¹H NMR: δ 7.71-7.68 (m, 4H), 7.40-7.36 (m, 6H), 3.65 (t, J=6.0 Hz, 2H), 3.50 (s, 4H), 2.60-2.40 (cm, 11H), 2.28 (s, 3H), 1.60-1.25 (cm, 44H), 1.09 (s, 9H), 0.90 (t, 6H). LRMS m/z 876 [M+H]⁺.

(xxxviii) 4-(methyl(6,8,26,28-tetrathiatritriacontan-17-yl)amino)butan-1-ol, 13. Pyridine-HF complex (70% w/w, 100 uL, excess) and pyridine (100 uL) were added to a

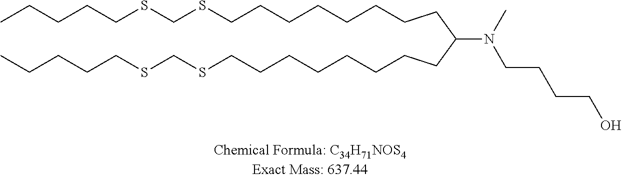 [SMILES: CCCCCSCSCCCCCCCCC(CCCCCCCCSCSCCCCC)N(C)CCCCO]

cold (0° C.) solution of 65 (100 mg, 114 umol) in dry THF (1 mL. The mixture was allowed to warm to room temperature and stirred overnight, then it was cooled back to 0° C. Aqueous saturated aq. NaHCO₃ solution (1 mL) and tert-butyl methyl ether (2 mL; tBuOMe reduces the extent of formation of a gelatinous precipitate and facilitates the extraction of the product) were added and the reaction mixture was extracted with diethyl ether (2×5 mL). The combined extracts were dried (Na₂SO₄), filtered and evaporated, and the residue was purified by flash chromatography (2% MeOH/DCM) to give 13 as a colorless oil (54 mg, 74%). ¹H NMR: δ 3.50 (s, 4H), 3.46 (t, 2H), 2.60-2.40 (cm, 11H), 2.28 (s, 3H), 1.60-1.25 (cm, 44H), 0.90 (t, 6H). LRMS m/z 638 [M+H]⁺.

(xxxix) (Chloromethyl)(octyl)sulfane, 68. Prepared from 1-octanethiol according to

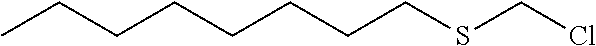

procedure (v) above. ¹H NMR (CDCl₃) δ: 4.75 (s, 2H), 2.78-2.70 (m, 2H), 1.65 (p, J=7.3 Hz, 2H), 1.45-1.20 (m, 10H), 0.92-0.84 (m, 3H).

(xl) (Chloromethyl)(nonyl)sulfane, 69. Prepared from 1-nonanethiol according to

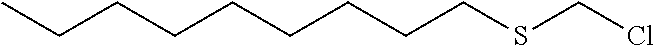

procedure (v) above. ¹H NMR (CDCl₃) δ: 4.75 (s, 2H), 2.74 (t, J=7.3 Hz, 2H), 1.65 (tt, J=7.6, 6.4 Hz, 2H), 1.40 (t, J=7.5 Hz, 2H), 1.35-1.21 (m, 10H), 0.88 (t, J=6.6 Hz, 3H). ¹³C NMR (CDCl₃) δ:50.1, 32.0, 31.8, 29.58, 29.4, 29.3, 28.9, 28.73, 22.8, 14.3.

(xli) Methyl 9-(((octylthio)methyl)thio)nonanoate, 70. Prepared from sulfane 68 and H₃C—(CH₂)₇—S—CH₂—S—CH₂—(CH₂)₇—COOMe thioacetate 41 according to procedure vi above. ¹H NMR (CDCl₃) δ: 3.66 (s, 3H), 3.65 (s, 2H), 2.65-2.58 (m, 4H), 2.29 (t, J=7.5 Hz, 2H), 1.65-1.51 (m, 6H), 1.43-1.24 (m, 18H), 0.91-0.83 (m, 3H). ¹³C NMR (CDCl₃) δ:174.4, 51.6, 35.5, 34.2, 31.9, 30.9, 30.9, 29.3, 29.2, 29.1, 29.1, 28.9, 25.0, 22.8, 14.2.

(xlii) Methyl 9-(((nonylthio)methyl)thio)nonanoate, 71. Prepared from sulfane 69 and H₃C—(CH₂)₈—S—CH₂—S—CH₂—(CH₂)₇—COOMe thioacetate 41 according to procedure vi above. ¹H NMR (CDCl₃) δ: 3.61 (s, 3H), 3.60 (s, 2H), 2.56 (t, J=7.4 Hz, 4H), 2.25 (t, J=7.5 Hz, 2H), 1.59-1.47 (m, 6H), 1.38-1.14 (m, 20H), 0.83 (t, J=6.8 Hz, 3H). ¹³C NMR (CDCl₃) δ:174.2, 51.4, 35.4, 34.0, 31.9, 30.8, 30.7, 29.5, 29.3, 29.2, 29.1 (3 peaks), 29.0, 28.9, 28.8, 24.9, 22.7, 14.1.

(xliii) Methyl 11-(((octylthio)methyl)thio)-2-(7-(((octylthio)methyl)thio)heptyl)-3-oxoundecanoate. Prepared from ester 70 according to procedure xiii above. ¹H NMR

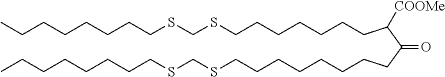 [SMILES: CCCCCCCCSCSCCCCCCCCC(=O)C(CCCCCCCSCSCCCCCCCC)C(=O)OC]

### (CDCl₃) δ: 3.75 (s, 3H), 3.68 (s, 4H), 2.64 (m, 8H), 2.59-2.43 (m, 1H), 1.89-1.79 (m, 2H), 1.71-1.21 (m, 52H), 0.94-0.86 (m, 6H).

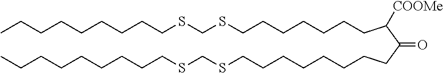 [SMILES: CCCCCCCCCSCSCCCCCCCCC(=O)C(CCCCCCCSCSCCCCCCCCC)C(=O)OC]

(xliv) Methyl 11-(((nonylthio)methyl)thio)-2-(7-(((nonylthio)methyl)thio)heptyl)-3-oxoundecanoate. Prepared from ester 71 according to procedure xiii above. ¹H NMR (CDCl₃) δ: 3.74 (s, 4H), 3.67 (s, 3H), 2.67-2.61 (m, 8H), 2.52 (qt, J=17.4, 7.3 Hz, 1H), 1.92-1.76 (m, 2H), 1.70-1.19 (m, 52H), 0.90 (t, J=6.4 Hz, 6H).

(xlv) 9,11,29,31-Tetrathianonatriacontan-20-one. Prepared from methyl 11-(((octylthio)methyl)thio)-2-(7-(((octylthio)methyl)thio)heptyl)-3-oxoundecanoate

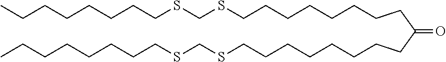 [SMILES: CCCCCCCCSCSCCCCCCCCC(=O)CCCCCCCCSCSCCCCCCCC]

according to procedure xvi above. ¹H NMR (400 MHz, CDCl₃) δ 3.65 (s, 4H), 2.65-2.57 (m, 8H), 2.37 (t, J=7.5 Hz, 4H), 1.63-1.49 (m, 12H), 1.41-1.20 (m, 36H), 0.87 (t, J=6.9 Hz, 6H).

(xlvi) 10,12,30,32-Tetrathiahentetracontan-21-one. Prepared from methyl 11-(((nonylthio)methyl)thio)-2-(7-(((nonylthio)methyl)thio)heptyl)-3-oxoundecanoate according to procedure xvi above. ¹H NMR (CDCl₃) δ 3.65 (s, 4H), 2.65-2.57 (m, 8H),

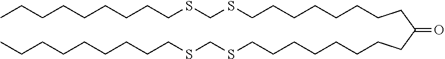 [SMILES: CCCCCCCCCSCSCCCCCCCCC(=O)CCCCCCCCSCSCCCCCCCCC]

2.38 (t, J=7.5 Hz, 4H), 1.57 (qd, J=8.1, 2.4 Hz, 12H), 1.43-1.19 (m, 40H), 0.87 (t, J=6.5 Hz, 6H). ¹³C NMR (CDCl₃) δ 211.7, 42.9, 35.5, 32.0, 31.0, 30.9, 29.6, 29.4 (3 peaks), 29.3, 29.2 (3 peaks), 29.1, 29.0, 24.0, 22.8, 14.3.

(xlvii) 2-(2,2-bis(8-(((Octylthio)methyl)thio)octyl)-1,3-dioxolan-4-yl)ethan-1-ol. Made from 9,11,29,31-tetrathianonatriacontan-20-one according to procedure xix above. ¹H

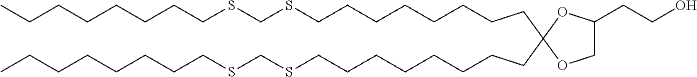 [SMILES: CCCCCCCCSCSCCCCCCCCC1(CCCCCCCCSCSCCCCCCCC)OCC(CCO)O1]

### NMR (400 MHz, CDCl₃) δ 4.26-4.18 (m, 1H), 4.07 (dd, J=7.9, 6.0 Hz, 1H), 3.82-3.76 (m, 2H), 3.64 (s, 4H), 3.52 (t, J=8.0 Hz, 1H), 2.61 (t, J=7.5 Hz, 8H), 1.86-1.75 (m, 2H), 1.66-1.49 (m, 14H), 1.45-1.21 (m, 38H), 0.95-0.82 (m, 6H).

(xlviii) 2-(2,2-bis(8-(((Nonylthio)methyl)thio)octyl)-1,3-dioxolan-4-yl)ethan-1-ol. Made from 10,12,30,32-tetrathiahentetracontan-21-one according to procedure xix above. ¹H

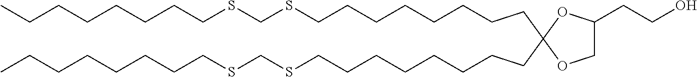 [SMILES: CCCCCCCCSCSCCCCCCCCC1(CCCCCCCCSCSCCCCCCCC)OCC(CCO)O1]

### NMR (CDCl₃) δ 4.26-4.18 (m, 1H), 4.07 (dd, J=7.9, 6.0 Hz, 1H), 3.82-3.76 (m, 2H), 3.64 (s, 4H), 3.52 (t, J=8.0 Hz, 1H), 2.61 (t, J=7.5 Hz, 8H), 1.86-1.75 (m, 2H), 1.66-1.49 (m, 14H), 1.45-1.21 (m, 42H), 0.95-0.82 (m, 6H).

(xlix) 2-(2,2-bis(8-(((Octylthio)methyl)thio)octyl)-1,3-dioxolan-4-yl)ethyl methanesulfonate. Prepared from 2-(2,2-bis(8-(((octylthio)methyl)thio)octyl)-1,3-dioxolan-4-yl)ethan-1-ol

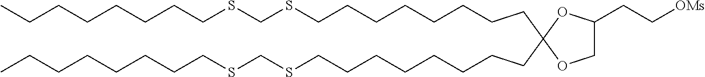 [SMILES: CCCCCCCCSCSCCCCCCCCC1(CCCCCCCCSCSCCCCCCCC)OCC(CCOS(C)(=O)=O)O1]

according to procedure xxii above. ¹H NMR (CDCl₃) δ 4.42-4.26 (m, 2H), 4.22-4.13 (m, 1H), 4.08 (dd, J=7.9, 6.1 Hz, 1H), 3.67 (s, 4H), 3.51 (t, J=7.7 Hz, 1H), 3.13 (s, 3H), 2.61 (t, J=7.5 Hz, 8H), 2.04-1.88 (m, 2H), 1.67-1.49 (m, 14H), 1.44-1.19 (m, 38H), 0.88 (t, J=7.1 Hz, 6H).

(xlx) 2-(2,2-bis(8-(((Nonylthio)methyl)thio)octyl)-1,3-dioxolan-4-yl)ethyl methanesulfonate. Prepared from 2-(2,2-bis(8-(((nonylthio)methyl)thio)octyl)-1,3-dioxolan-4-yl)ethan-1-ol according to procedure xxii above. ¹H NMR (CDCl₃) δ 4.42-4.26 (m, 2H), 4.22 -4.13

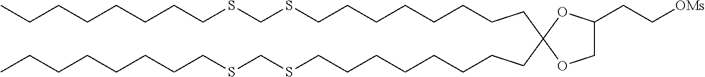 [SMILES: CCCCCCCCSCSCCCCCCCCC1(CCCCCCCCSCSCCCCCCCC)OCC(CCOS(C)(=O)=O)O1]

### (m, 1H), 4.08 (dd, J=7.9, 6.1 Hz, 1H), 3.67 (s, 4H), 3.51 (t, J=7.7 Hz, 1H), 3.13 (s, 3H), 2.61 (t, J=7.5 Hz, 8H), 2.04-1.88 (m, 2H), 1.67-1.49 (m, 14H), 1.44-1.19 (m, 42H), 0.88 (t, J=7.1 Hz, 6H).

(xlxi) 2-(2,2-bis(8-(((octylthio)methyl)thio)octyl)-1,3-dioxolan-4-yl)-N,N-dimethyl-ethan-1-amine (7). Prepared from 2-(2,2-bis(8-(((Octylthio)methyl)thio)octyl)-1,3-dioxolan-4-yl)ethyl methanesulfonate according to procedure xxv above. ¹H NMR (C₆D₆)

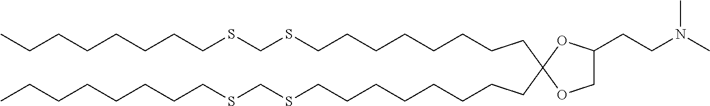 [SMILES: CCCCCCCCSCSCCCCCCCCC1(CCCCCCCCSCSCCCCCCCC)OCC(CCN(C)C)O1]

δ 4.10 (t, J=6.6 Hz, 1H), 3.95 (dd, J=7.8, 5.9 Hz, 1H), 3.50 (s, 4H), 3.44 (t, J=7.8 Hz, 1H), 2.61-2.49 (m, 8H), 2.37-2.19 (m, 2H), 2.02 (s, 6H), 1.87-1.74 (m, 4H), 1.68-1.44 (m, 12H), 1.42-1.11 (m, 38H), 0.83 (t, J=7.1 Hz, 6H). LRMS (ESI+) m/z 750 [M+H]⁺.

(xlxii) 2-(2,2-bis(8-(((nonylthio)methyl)thio)octyl)-1,3-dioxolan-4-yl)-N,N-dimethyl-ethan-1-amine (8). Prepared from 2-(2,2-bis(8-(((nonylthio)methyl)thio)octyl)-1,3-dioxolan-4-yl)ethyl

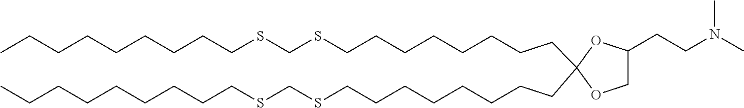 [SMILES: CCCCCCCCCSCSCCCCCCCCC1(CCCCCCCCSCSCCCCCCCCC)OCC(CCN(C)C)O1]

methanesulfonate according to procedure xxv above. ¹H NMR (C₆D₆) δ 4.10 (t, J=6.6 Hz, 1H), 3.95 (dd, J=7.8, 5.9 Hz, 1H), 3.50 (s, 4H), 3.44 (t, J=7.8 Hz, 1H), 2.61-2.49 (m, 8H), 2.37-2.19 (m, 2H), 2.02 (s, 6H), 1.87-1.74 (m, 4H), 1.68-1.44 (m, 12H), 1.42-1.11 (m, 42H), 0.83 (t, J=7.1 Hz, 6H). LRMS (ESI+) m/z 778 [M+H]⁺.

(xlxiii) Methyl 6-hydroxyhexanoate, 73. Prepared from monomethyl adipate according to

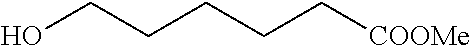

procedure ii above. ¹H NMR (CDCl₃) δ 3.64 (s, 3H), 3.60 (t, J=6.8 Hz, 2H), 2.27 (t, J=1.2 Hz, 2H), 1.62-1.28 (m, 6H).

(xlxiv) 9,11,23,25-tetrathiatritriacontan-17-one, 74. Prepared from methyl 6-hydroxyhexanoate

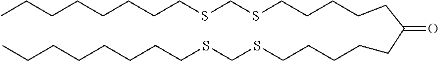 [SMILES: CCCCCCCCSCSCCCCCC(=O)CCCCCSCSCCCCCCCC]

according to procedures iii, iv, vi (but with sulfane 68), xii, and xiv above. ¹H NMR (CDCl₃) δ 3.65 (s, 4H), 2.62 (t, J=7.4 Hz, 4H), 2.61 (t, J=7.4 Hz, 4H), 2.40 (t, J=7.4 Hz, 4H), 1.65-1.51 (m, 12H), 1.44-1.20 (m, 24H), 0.88 (t, J=6.6 Hz, 6H). ¹³C NMR (CDCl₃) δ 211.1, 42.8, 35.54, 32.0, 31.0, 30.7, 29.3, 29.2, 29.1, 29.00, 28.6, 23.5, 22.8, 14.3.

(xlxv) 2-(2,2-bis(5-(((Octylthio)methyl)thio)pentyl)-1,3-dioxolan-4-yl)-N,N-dimethyl-ethan-1-amine, 9. Prepared from 74 according to procedures xix, xxii, and xxv above. ¹H

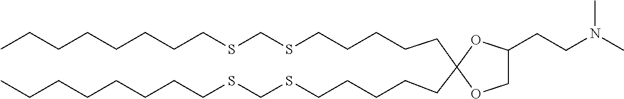 [SMILES: CCCCCCCCSCSCCCCCC1(CCCCCSCSCCCCCCCC)OCC(CCN(C)C)O1]

NMR (C₆D₆) δ 4.11 (t, J=6.6 Hz, 1H), 3.94 (dd, J=7.8, 5.9 Hz, 1H), 3.51 (s, 4H), 3.44 (t, J=7.8 Hz, 1H), 2.61-2.49 (m, 8H), 2.37-2.19 (m, 2H), 2.02 (s, 6H), 1.87-1.74 (m, 4H), 1.68-1.44 (m, 12H), 1.42-1.11 (m, 26H), 0.83 (t, J=7.1 Hz, 6H). LRMS (ESI+) m/z 666 [M+H]⁺.

(xlxvi) 6,8,26,28-tetrathiatritriacontan-17-ol, 75. Solid NaBH₄ (50 mg, 1.3 mmol) was added portionwise to a cold (0° C.), stirred solution of ketone 54 (1.1 g, 2.0 mmol, 1 equiv)

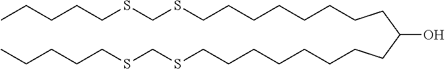 [SMILES: CCCCCSCSCCCCCCCCC(O)CCCCCCCCSCSCCCCC]

in 95% ethanol (10 mL). After stirring at 0° C. for 1 h, the reaction was checked for completion, either by TCL (5% ether in hexanes) or, more reliably, by adding 1 drop of the reaction mixture to saturated aqueous NH₄Cl solution (0.5 mL), extracting with hexanes, evaporating the combined extracts to dryness, and checking the residue by ¹H NMR. Additional NaBH₄ was added if the reaction was found to be incomplete. Upon completion, the reaction was quenched by careful addition of aqueous saturated NH₄Cl solution (caution should be taken due to H₂ evolution and foaming) and concentrated on the rotary evaporator to remove the ethanol. The aqueous residue was extracted with hexanes (3×10 mL). The combined extracts were passed through a plug of anhydrous Na₂SO₄ and concentrated to afford crude alcohol, which was purified by silica gel column chromatography with 5→10% v/v ethyl acetate in hexanes to afford pure product (1.0 g, 91%). ¹H NMR (CDCl₃): δ 3.66 (s, 4H), 3.58 (m, 1H), 2.63 (t, J=7.3 Hz, 8H), 1.62-1.55 (m, 10H), 1.38-1.26 (m, 30H), 0.91 (t, J=6.6 Hz, 6H) ppm. ¹³C NMR (75 MHz, CDCl₃): δ 72.0, 37.5, 35.4, 31.1, 30.8, 29.6, 29.5, 29.2, 29.1, 28.9, 28.8, 25.7, 22.3, 13.9.

(xlxvii) 4,8,26,30-tetrathiatritriacontan-17-ol, 76. Prepared from ketone 56 according to procedure xlxvi above. ¹H NMR: δ 3.57 (m, 1H), 2.60 (t, J=7.2 Hz, 8H), 2.49 (t, J=7.0

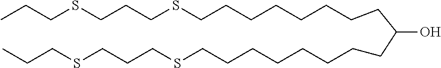 [SMILES: CCCSCCCSCCCCCCCCC(O)CCCCCCCCSCCCSCCC]

### Hz, 8H), 1.90-1.80 (m, 4H), 1.67-1.52 (m, 9H), 1.42-1.29 (m, 24H), 0.98 (t, J=7.2 Hz, 6H) ppm. ¹³C NMR: δ 71.9, 37.5, 34.2, 32.1, 31.0, 30.9, 29.6, 29.5, 29.4, 29.2, 28.9, 25.6, 22.9, 13.5.

(xlxviii) 4-((tert-Butyldiphenylsilyl)oxy)-N-methylbutan-1-amine. A solution of tert-butyl(chloro)-diphenylsilane (2.9 g, 10.7 mmol, 1.1 equiv) in CH₂Cl₂ (2 mL) was added

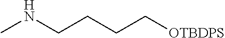

dropwise during 15 min to a well-stirred solution of 4-(methylamino)-1-butanol (1.0 g, 9.7 mmol, 1.0 equiv) and imidazole (1.5 g, 21.3 mmol, 2.2 equiv) in DCM (4 mL). The mixture was stirred overnight at room temperature. The reaction mixture was sequentially washed with sat. aq. NaHCO₃ solution (2×5 mL), water (2×5 mL), and sat. aq. NaCl chloride solution (2×5 mL), then dried over anhydrous Na₂SO₄, filtered, and concentrated under reduced pressure to furnish 55 (3.1 g, 94%) as a yellow oil. ¹H NMR: δ 7.71-7.68 (m, 4H), 7.40-7.36 (m, 6H), 3.72 (t, J=6.0 Hz, 2H), 2.67 (t, J=6.6 Hz, 2H), 2.50 (s, 3H), 1.65-1.48 (m, 4H), 1.09 (s, 9H). LRMS m/z 342 [M+H]⁺.

(xlxxix) Ethyl 4-((4-((tert-butyldiphenylsilyl)oxy)butyl)(methyl)amino)butanoate. A solution of ethyl 4-bromobutyrate (250 mg, 1.3 mmol) and 4-((tert-butyldiphenylsilyl)oxy)-N-methylbutan-1-amine

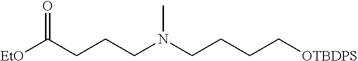

(341 mg, 1.0 mmol) in MeOH (8 mL) in a sealed microwave reaction vial was microwave irradiated for 15 minutes (normal absorption, 115° C.), then it was cooled to room temperature and concentrated. The residue was purified by silica gel column chromatography (0-20% MeOH in DCM) to yield the desired product (342 mg, 75%). ¹H NMR: δ 7.71-7.68 (m, 4H), 7.40-7.36 (m, 6H), 4.12 (q, J=6.5 Hz, 2H), 3.72 (t, J=6.0 Hz, 2H), 2.50-2.43 (m, 6H), 2.40 (s, 3H) 1.86-1.48 (m, 6H), 1.09 (s, 9H), 1.07 (t, J=6.5 Hz, 3H). LRMS m/z 456 [M+H]⁺.

(l) 4-((4-((tert-Butyldiphenylsilyl)oxy)butyl)(methyl)amino)butanoic acid hydro-chloride. A solution of ethyl 4-((4-((tert-butyldiphenylsilyl)oxy)butyl)(methyl)-amino)butanoate (200 mg, 440 umol) and aqueous 1 N LiOH (1 ml, 1 mmol) in THF (2 mL)

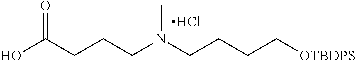

was stirred at room temperature overnight, whereupon saponification was complete. The solution was cooled to 0° C. and acidified to pH 4 with 1 N HCl, then it was concentrated. The aqueous residue was extracted with ethyl acetate (3×2 mL) and the combined extracts were filtered over a plug of Na₂SO₄ and concentrated to dryness. The crude acid 60 thus obtained appeared to contain some inorganic matter, but it was used directly in the next step without purification. LRMS m/z 428 [M+H]⁺.

(li) 6,8,26,28-Tetrathiatritriacontan-17-yl 4-((4-((tert-butyldiphenylsilyl) oxy)butyl)(methyl)amino)butanoate. A solution of alcohol 75 (50.0 mg, 90 umol), crude 4-((4-((tert-butyldiphenylsilyl)oxy)butyl)(methyl)amino)butanoic acid hydro-chloride (150

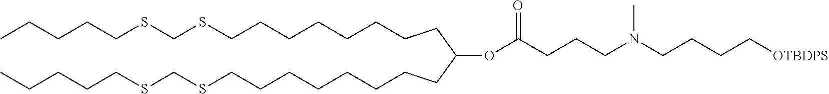

EDCI—HCl (22 mg, 110 umol) and DMAP (18 mg, 147 umol) in CH₂Cl₂ (2 mL) was stirred at room temperature for 18 hours under a nitrogen atmosphere. The mixture was concentrated and the residue was purified by silica gel column chromatography (0-5% MeOH in DCM) to yield the desired product (35 mg, 40%). ¹H NMR: δ 7.71-7.68 (m, 4H), 7.40-7.36 (m, 6H), 4.85 (m, 1H), 3.72 (t, J=6.0 Hz, 2H), 3.65 (s, 4H), 2.60-2.40 (m, 14H), 2.40 (br s, 3H), 2.38-1.30 (cm, 46H), 1.09 (s, 9H), 0.89 (t, 6H). LRMS m/z 962 [M+H]⁺.

(lii) 6,8,26,28-Tetrathiatritriacontan-17-yl 4-((4-hydroxybutyl)(methyl)amino) butanoate, 14. Pyridine-HF complex (70% w/w, 50 uL, excess) and pyridine (50 uL) were added to a cold (0° C.) solution of 6,8,26,28-tetrathiatritriacontan-17-yl 4-((4-((tert-butyldiphenylsilyl) oxy)butyl)(methyl)amino)butanoate (30 mg, 31 umol, 1.0 equiv) in 0.5

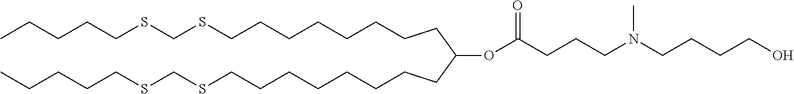 [SMILES: CCCCCSCSCCCCCCCCC(CCCCCCCCSCSCCCCC)OC(=O)CCCN(C)CCCCO]

mL of anhydrous THF. The mixture was allowed to warm to room temperature and stirred overnight, then it was cooled back to 0° C. Aqueous saturated aq. NaHCO₃ solution (0.5 mL) and tert-butyl methyl ether (1 mL; tBuOMe reduces the extent of formation of a gelatinous precipitate and facilitates the extraction of the product) were added and the reaction mixture was extracted with diethyl ether (2×5 mL). The combined extracts were dried (Na₂SO₄), filtered and evaporated, and the residue was purified by flash chromatography (2% MeOH/DCM) to give 25 as a colorless oil (16 mg, 70%). ¹H NMR: δ 4.85 (m, 1H), 3.68 (t, J=6.0 Hz, 2H), 3.65 (s, 4H), 2.60-2.40 (m, 14H), 2.40 (br s, 3H), 2.38-1.30 (cm, 46H), 0.89 (t, 6H). LRMS m/z 724 [M+H]⁺.

(liii) 3-(4-(2-Hydroxyethyl)-1,3-dioxolan-2-yl)propanenitrile. A solution of 4,4-dimethoxybutyronitrile

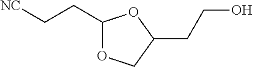 [SMILES: N#CCCC1OCC(CCO)O1]

(250 mg, 1.94 mmol), 1,2,4-butanetriol (411 mg, 3.88 mmol), and concentrated H₂SO₄ (0.01 mL) in 1,4 dioxane (7.00 mL) was heated to 80° C. for 4 hours. The mixture was then cooled, diluted with water (5.00) and extracted with CH₂Cl₂ (3×5.00 mL). The combined extracts were dried (Na₂SO₄) and concentrated to yield the product (341 mg, crude) which was used in the next step without purification. ¹H NMR (CDCl₃) δ 4.74 (t, J=4.6 Hz, 1H), 4.14 (ddd, J=11.4, 5.2, 1.4 Hz, 1H), 3.80-3.72 (m, 2H), 3.66 (dd, J=11.8, 3.2 Hz, 1H), 3.61-3.55 (m, 1H), 2.56-2.38 (m, 2H), 2.02-1.92 (m, 2H), 1.89-1.72 (m, 1H), 1.44-1.34 (m, 1H).

(liv) 2-(2-(2-Cyanoethyl)-1,3-dioxolan-4-yl)ethyl 4-toluenesulfonate. A solution of 3-(4-(2-hydroxyethyl)-1,3-dioxolan-2-yl)propanenitrile

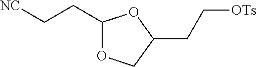 [SMILES: Cc1ccc(S(=O)(=O)OCCC2COC(CCC#N)O2)cc1]

(341 mg, crude), Et₃N (416 uL, 3.0 mmol), and TsCl (456 mg, 2.4 mmol) in CH₂Cl₂ (8 mL) was stirred at room temperature for 18 hours under a nitrogen atmosphere. The mixture was then diluted with water (10 mL) and extracted with CH₂Cl₂ (3×10 mL). The combined extracts were dried (Na₂SO₄) and concentrated and the residue was purified by silica gel column chromatography (0-15% MeOH in CH₂Cl₂) to yield 497 mg of the desired tosylate (79% over 2 steps). ¹H NMR (CDCl₃) δ 7.79 (d, J=8.4 Hz, 2H), 7.45-7.32 (m, 2H), 4.62 (t, J=4.6 Hz, 1H), 4.11 (ddd, J=11.5, 5.0, 1.4 Hz, 1H), 4.05-3.97 (m, 2H), 3.96-3.88 (m, 1H), 3.72 (td, J=12.0, 2.6 Hz, 1H), 2.46 (s, 3H), 2.37 (td, J=7.5, 4.8 Hz, 2H), 1.87 (td, J=7.6, 4.7 Hz, 2H), 1.72-1.57 (m, 1H), 1.46-1.38 (m, 1H).

(lv) 3-(4-(2-(Dimethylamino)ethyl)-1,3-dioxolan-2-yl)propanenitrile. A solution of 2-(2-(2-cyanoethyl)-1,3-dioxolan-4-yl)ethyl

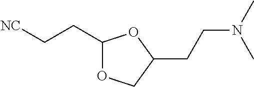

4-toluenesulfonate (497 mg, 1.5 mmol) and Me₂H (2N in THF, 8 mL) in MeOH (8 mL) in a sealed microwave reaction vial was microwave irradiated for 15 minutes (normal absorption, 115° C.), then it was cooled to room temperature and concentrated. The residue was purified silica gel column chromatography (0-20% MeOH in DCM) to yield the product (215 mg, 71%). ¹H NMR: δ 4.72 (t, J=4.5 Hz, 1H), 4.25-4.15 (m, 1H), 4.10 (ddd, J=11.5, 5.0, 1.4 Hz, 1H), 3.76 (td, J=11.9, 2.7 Hz, 1H), 2.88-2.82 (m, 2H), 2.68 (s, 6H), 2.58-2.37 (m, 2H), 1.93 (m, 2H), 1.64-1.44 (m, 2H).

(lvi) 3-(4-(2-(dimethylamino)ethyl)-1,3-dioxolan-2-yl)propanoic acid hydrochloride. A solution of 3-(4-(2-(dimethylamino)ethyl)-1,3-dioxolan-2-yl)propanenitrile (215 mg, 1.1

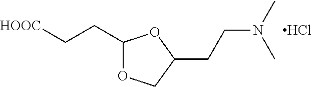

mmol) in EtOH (5.00 mL) containing 6 N aqueous NaOH (2.5 mL) was heated at reflux for 18 hours, then it was cooled to room temperature and acidified to pH 6 with 6 N HCl. The mixture was concentrated to yield the hydrochloride of the acid as a crude mixture containing inorganic salts. This mixture was used directly in the next step without purification. ¹H NMR (400 MHz, D₂O) δ 4.91 (t, J=5.3 Hz, 1H), 4.41 (d, J=3.4 Hz, 1H), 4.28 (dd, J=11.6, 4.7 Hz, 1H), 4.04 (td, J=11.7, 3.3 Hz, 1H), 3.40-3.38 (m, 2H), 3.05 (s, 6H), 2.38 (td, J=7.5, 4.5 Hz, 2H), 2.07-1.92 (m, 2H), 1.88-1.69 (m, 2H).

(lvii) 6,8,26,28-Tetrathiatritriacontan-17-yl 3-(4-(2-(dimethylamino)ethyl)-1,3-dioxolan-2-yl)propanoate, 15. A solution of alcohol 75 (50.0 mg, 90 umol), crude 3-(4-(2-(dimethylamino)ethyl)-1,3-dioxolan-2-yl)propanoic acid hydrochloride (150 mg), EDCI—HCl

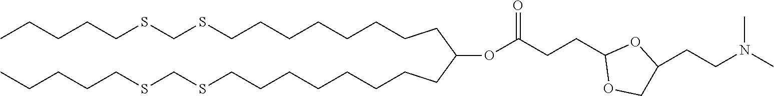 [SMILES: CCCCCSCSCCCCCCCCC(CCCCCCCCSCSCCCCC)OC(=O)CCC1OCC(CCN(C)C)O1]

(22 mg, 110 umol) and DMAP (18 mg, 147 umol) in CH₂Cl₂ (2 mL) was stirred at room temperature for 18 hours under a nitrogen atmosphere. The mixture was then concentrated, and the residue was purified by silica gel column chromatography (0-5% MeOH in DCM) to yield lipid 15 (24 mg, 36%). ¹H NMR (CDCl) δ 4.85 (p, J=6.0 Hz, 1H), 4.61 (t, J=4.9 Hz, 1H), 4.09 (dd, J=11.4, 4.9 Hz, 1H), 3.84 (m, 1H), 3.73 (td, J=11.9, 2.6 Hz, 1H), 3.66 (s, 4H), 2.63 (t, J=7.3 Hz, 8H), 2.43 (m, 2H), 2.20 (br s, 6H), 1.95 (m, 2H), 1.63-1.55 (m, 12H), 1.38-1.26 (m, 32H), 0.91 (t, J=6.6 Hz, 6H). LRMS m/z 752 [M+H]⁺.

(lviii) 3-(4-(2-((3-Hydroxypropyl)(methyl)amino)ethyl)-1,3-dioxolan-2-yl)propane-nitrile. A solution of 2-(2-(2-cyanoethyl)-1,3-dioxolan-4-yl)ethyl 4-toluenesulfonate (497

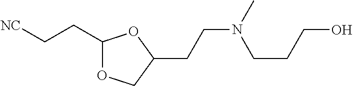 [SMILES: CN(CCCO)CCC1COC(CCC#N)O1]

mg, 1.5 mmol) and 3-(methylamino)-1-propanol (270 mg, 3 mmol) in MeOH (8 mL) in a sealed microwave reaction vial was microwave irradiated for 15 minutes (normal absorption, 115° C.), then it was cooled to room temperature and concentrated. The residue was purified silica gel column chromatography (0-20% MeOH in DCM) to yield the product (300 mg, 82%). ¹H NMR: δ 4.73 (t, J=4.5 Hz, 1H), 4.25-4.15 (m, 1H), 4.10 (ddd, J=11.5, 5.0, 1.4 Hz, 1H), 3.78 (td, J=11.9, 2.7 Hz, 1H), 3.73 (t, J=6.9 Hz, 2H), 2.88-2.82 (m, 4H), 2.70 (s, 3H), 2.58-2.37 (m, 4H), 1.93 (m, 2H), 1.70-1.44 (m, 2H). LRMS m/z 243 [M+H]⁺s.

(lix) 3-(4-(2-((3-Hydroxypropyl)(methyl)amino)ethyl)-1,3-dioxolan-2-yl)propanoic acid hydrochloride. A solution of 3-(4-(2-((3-hydroxypropyl)(methyl)amino)ethyl)-1,3-dioxolan-2-yl)propane-nitrile

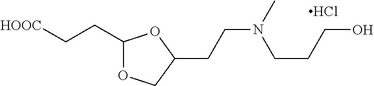 [SMILES: CN(CCCO)CCC1COC(CCC(=O)O)O1 ; Cl]

(300 mg, 1.23 mmol) in EtOH (5.00 mL) containing 6 N aqueous NaOH (2.5 mL) was heated at reflux for 18 hours, then it was cooled to room temperature and acidified to pH 6 with 6 N HCl. The mixture was concentrated to yield the crude acid, which contained inorganic salts. This mixture was used directly in the next step without purification. ¹H NMR (D₂O): δ 4.90 (t, J=5.3 Hz, 1H), 4.41 (d, J=3.4 Hz, 1H), 4.28 (dd, J=11.6, 4.7 Hz, 1H), 4.04 (td, J=11.7, 3.3 Hz, 1H), 3.73 (t, J=6.9 Hz, 2H), 3.40-3.38 (m, 4H), 3.10 (s, 3H), 2.38 (m, 2H), 2.07-1.92 (m, 2H), 1.88-1.69 (m, 4H).

(lx) 3-(4-(2-(Methyl(3-((triethylsilyl)oxy)propyl)amino)ethyl)-1,3-dioxolan-2-yl)propanoic acid hydrochloride. Crude 3-(4-(2-((3-hydroxypropyl)(methyl)amino)ethyl)-1,3-dioxolan-2-yl)propanoic acid hydrochloride was

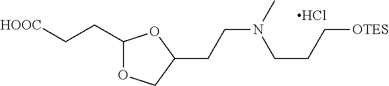

thoroughly dried under vacuum, then it was taken up in pyridine (2 mL) and treated with imidazole (82 mg, 1.2 mmol) and Et₃SiCl (225 mg, 1.5 mmol). The mixture was stirred overnight at room temperature, then it was evaporated to dryness and the residue was taken up in water (2 mL). The solution was stirred at room temperature for 1 h, then it was acidified to pH 6 and evaporated to dryness. The residue of was taken up with CH₂Cl₂ and the organic phase was filtered and evaporated to yield the crude acid, which was thoroughly dried under vacuum and used in the next step without purification. ¹H NMR (CDCl₃): δ 4.87 (t, J=5.3 Hz, 1H), 4.39 (d, J=3.4 Hz, 1H), 4.28 (dd, J=11.6, 4.7 Hz, 1H), 4.04 (td, J=11.7, 3.3 Hz, 1H), 3.68 (t, J=6.9 Hz, 2H), 3.40-3.38 (m, 4H), 3.10 (s, 3H), 2.38 (m, 2H), 2.07-1.92 (m, 2H), 1.88-1.69 (m, 4H), 0.99 (t, 8H), 0.70 (br q, 6H).

(lxi) 4,8,26,30-Tetrathiatritriacontan-17-yl 3-(4-(2-(methyl(3-((triethylsilyl)oxy)

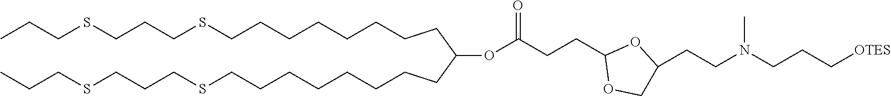

propyl)amino)ethyl)-1,3-dioxolan-2-yl)propanoate. Prepared from alcohol 76 and crude 3-(4-(2-(methyl(3-((triethylsilyl)oxy)propyl)amino)ethyl)-1,3-dioxolan-2-yl)propanoic acid hydrochloride according to procedure xxxiii above. ¹H NMR: δ 4.87 (t, J=5.3 Hz, 1H), 4.83 (quin, J=6.3 Hz, 1H), 4.39 (d, J=3.4 Hz, 1H), 4.28 (dd, J=11.6, 4.7 Hz, 1H), 4.04 (dt, J=11.7, 3.3 Hz, 1H), 3.68 (t, J=6.9 Hz, 2H), 3.40-3.38 (m, 4H), 2.60 (t, J=7.2 Hz, 8H), 2.49 (t, J=7.0 Hz, 8H), 2.38 (m, 2H), 2.11-1.69 (m, 14H), 1.67-1.52 (m, 9H), 1.42 1.29 (m, 22H), 0.98 (br t, 15H), 0.70 (br q, 6H). LRMS m/z 910 [M+H]⁺.

(lxii) 4,8,26,30-Tetrathiatritriacontan-17-yl 3-(4-(2-((3-hydroxypropyl)(methyl) amino)ethyl)-1,3-dioxolan-2-yl)propanoate, 16. Prepared in 73% yield from 4,8,26,30-Tetrathiatritriacontan-17-yl 3-(4-(2-(methyl(3-((triethylsilyl)oxy) propyl)amino)ethyl)-1,3-dioxolan-2-yl)propanoate according to procedure xxxiv above. ¹H NMR: δ 4.87 (t, J=5.3

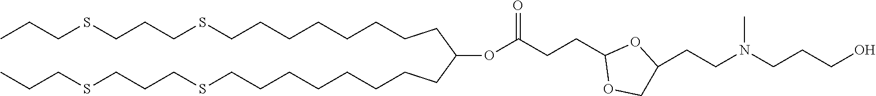 [SMILES: CCCSCCCSCCCCCCCCC(CCCCCCCCSCCCSCCC)OC(=O)CCC1OCC(CCN(C)CCCO)O1]

Hz, 1H), 4.84 (quin, J=6.3 Hz, 1H), 4.39 (d, J=3.4 Hz, 1H), 4.28 (dd, J=11.6, 4.7 Hz, 1H), 4.04 (dt, J=11.7, 3.3 Hz, 1H), 3.66 (t, J=6.9 Hz, 2H), 3.40-3.38 (m, 4H), 11 2.60 (t, J=7.2 Hz, 8H), 2.49 (t, J=7.0 Hz, 8H), 2.38 (m, 2H), 2.11-1.69 (m, 14H), 1.67-1.52 (m, 9H), 1.42-1.29 (m, 22H), 0.99 (br t, 6H). LRMS m/z 796 [M+H]⁺.

(lxiii) 10-(((Pentylthio)methyl)thio)decan-1-ol, 80. A solution of ester 44 (1.2 g, 3.7

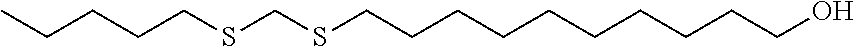 [SMILES: CCCCCSCSCCCCCCCCCCO]

mmol) in THF (2 mL) was added dropwise to a cold (0° C.), well stirred suspension of LAH (170 mg, 4.5 mmol, 1.2 equiv) in 4 mL of THF, under nitrogen. The mixture was allowed to warm to room temperature and after 3 h it was quenched by sequential addition of 170 μL of H₂O, 170 μL of 6M NaOH, and 510 μL of H₂O. The mixture was extracted with ether (5×10 mL). The combined extracts were dried (Na₂SO₄) and concentrated to afford 1.03 g of crude 80 (95% yield), which was used without purification. ¹H NMR (CDCl₃) δ 3.6 (s, 3H, overlapping with t, 2H), 2.6 (t, J=7.4 Hz, 4H), 1.7-1.5 (m, 7H), 1.5-1.2 (m, 14H), 1.0-0.8 (m, 3H). ¹³C NMR (CDCl₃) δ 77.4, 63.2, 35.6, 32.9, 31.7, 31.2, 31.0, 29.6, 29.5, 29.3, 29.2, 29.0, 28.9, 25.9, 22.8, 22.8, 22.4, 14.3, 14.1.

(lxiv) 10-(((Pentylthio)methyl)thio)decyl methanesulfonate, 81. Prepared from alcohol

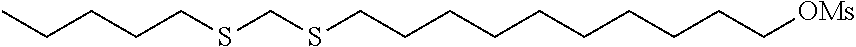 [SMILES: CCCCCSCSCCCCCCCCCCOS(C)(=O)=O]

80 according to procedure iii above. ¹H NMR (300 MHz, CDCl₃) δ 4.2 (t, J=6.6 Hz, 2H), 3.6 (s, 3H), 3.0 (s, 3H), 2.6 (t, J=7.4 Hz, 4H), 1.8-1.7 (m, 2H), 1.6-1.4 (m, 4H), 1.4-1.1 (m, 12H), 0.9 (t, J=6.9 Hz, 3H). ¹³C NMR (75 MHz, CDCl₃) δ 70.3, 37.5, 35.5, 31.2, 30.9, 30.9, 29.4, 29.2, 29.2, 29.1, 29.1, 28.9, 28.9, 25.5, 22.4, 14.1.

(lxv) 4-(bis(9-(((Pentylthio)methyl)thio)nonyl)amino)butan-1-ol, 17. A solution of 4-amino-1-butanol

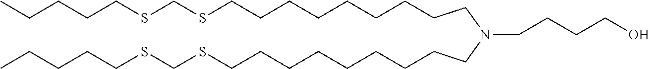 [SMILES: CCCCCSCSCCCCCCCCCN(CCCCO)CCCCCCCCCSCSCCCCC]

(21 mg, 230 umol, 1 equiv) and mesylate 81 (200 mg, 540 umol, 2.3 equiv) in dry acetonitrile (1 mL) containing suspended anhydrous Na₂CO₃ (50 mg, 470 umol, 2.0 equiv) and NaI (34 mg, 230 umol, 1 equiv) was heated to 75° C. in a sealed reactor, under argon atmosphere. The reaction was monitored by TLC, MS and ¹H NMR. A lower layer immiscible with MeCN and containing the desired product separated. After 18 hours, the mixture was cooled concentrated under vacuum. The residue was partitioned between hexane (5 mL) and water (5 mL). The organic phase was separated and the aqueous layer was extracted with more hexane (2×5 mL). The combined extracts were washed with brine (2×5 mL), then dried (Na₂SO₄) and evaporated. The residue was purified by column chromatography (5→7% MeOH in CH₂Cl₂) to afford 70 mg (48%) of pure product. ¹H NMR (400 MHz, CDCl₃) δ 3.7-3.6 (m, 6H), 2.8-2.7 (m, 6H), 2.6 (t, J=7.7 Hz, 8H), 1.8-1.7 (m, 2H), 1.7-1.4 (m, 13H), 1.4-1.1 (m, 30H), 0.9 (t, J=7.0 Hz, 6H). ¹³C NMR (101 MHz, CDCl₃) δ 62.0, 52.9, 35.5, 31.2, 30.9, 30.9, 29.3, 29.2, 28.9, 28.9, 27.3, 22.4, 14.1. LRMS m/z 638 [M+H]⁺.

### Example 2: MRNA-Containing LNPs Comprising a Sulfur-Containing Ionizable Lipid of Formula A Exhibit Improved In Vivo Extrahepatic Delivery of mRNA

The in vivo transfection of the following firefly luciferase mRNA formulations (reported in mol %) comprising nor-MC3 or ionizable sulfur-containing lipid 4 [chemical name: 2-(2,2-bis(8-(((pentylthio)methyl)thio)octyl)-1,3-dioxolan-4-yl)-N,N-dimethylethan-1-amine], DSPC, cholesterol and PEG₂₀₀₀-DMG was compared in this example.

TABLE 1
Formulations examined in vivo containing firefly luciferase mRNA

| Ionizable lipid | Lipid composition/mol % |
|---|---|
| norMC3 | norMC3:DSPC:Chol:PEG₂₀₀₀-DMG (50:10:38.5:1.5) |
| 4 (MF020) | Lipid 4 (MF020):DSPC:Chol:PEG₂₀₀₀-DMG |
| | (50:10:38.5:1.5) |

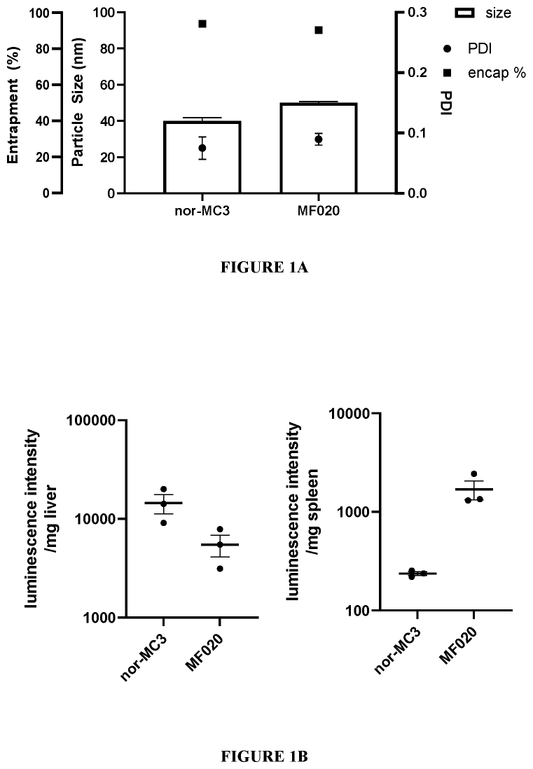

The biophysical characteristics of the formulations in Table 1 were analyzed as described in the Methods above. The nor-MC3 formulation had a size of about 39.9 nm, a polydispersity index (PDI) of about 0.075 and an entrapment efficiency of 94%. The inventive formulation containing 50 mol % 4 had a slightly larger size of about 50.0 nm, a PDI of about 0.090 and an encapsulation efficiency approaching 90%. The results are presented in FIG. 1A.

The in vivo transfection of the mRNA-LNP formulations of Table 1 was carried out as described in the Methods above and the results are shown in FIG. 1B. The left panel of FIG. 1B compares the luminescence intensity/mg of liver for the norMC3 LNP formulation versus the sulfur-containing lipid 4 of the disclosure, and the right panel compares the luminescence intensity of the same two formulations in the spleen. The results show that the inventive LNP formulation exhibited a higher luminescence intensity in the spleen (signal/per mg liver or spleen) relative to the nor-MC3 formulation. The trend was reversed in the liver. This demonstrates that formulations comprising a sulfur-containing lipid of the disclosure (e.g., encompassed by Formula A) have improved extrahepatic delivery relative to an otherwise identical formulation comprising an ionizable, norMC3 lipid lacking the sulfur atoms in the lipid chains.

Although the invention has been described and illustrated with reference to the foregoing detailed description and examples, it will be apparent that a variety of modifications and changes may be made without departing from the invention.

1. A lipid having the structure of Formula A: 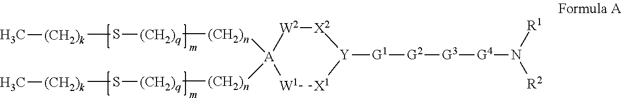
or a salt thereof,
wherein:
each k independently is 1 to 10;
each m independently is 1 to 4;
each n independently is 1 to 10; and
each q independently is 1 to 5,
A is C or N, and
if A is C, then
W¹ and X¹ are bonded to each other as indicated by the dashed bond, and
W¹ is O or S;
W² is 0 or S;
X¹ is (CH₂)ₘ, wherein m is 1 or 2;
X² is (CH₂)ₙ, wherein n is 0 to 2;
Y is CH; and
G¹ is (CH₂)ᵣ, wherein r is 1 to 6;
G²-G³-G⁴ is either present or absent, and
if G²-G³-G⁴ is present, then
G² is O;
G³ is a carbonyl group;
G⁴ is (CH₂)ₛ, wherein s is 1 to 6;
R¹ is a C₁-C₃ alkyl; and
R² is a C₁-C₆ alkyl optionally bearing an OH substituent;
if G²-G³-G⁴ is absent, then NR¹R² is directly bonded to G¹, and
R¹ is a C₁-C₃ alkyl; and
R² is a C₁-C₆ alkyl optionally bearing an OH substituent; and
if A is N, then
W¹ and X¹ are not bonded to each other,
W², X¹, X², Y, G¹, G², G³, G⁴ and —NR¹R² are absent, and
W¹ is (CH₂)ₜ—OH, wherein t is 1 to 6.

2. The lipid or the salt thereof of claim 1, selected from the group consisting of:

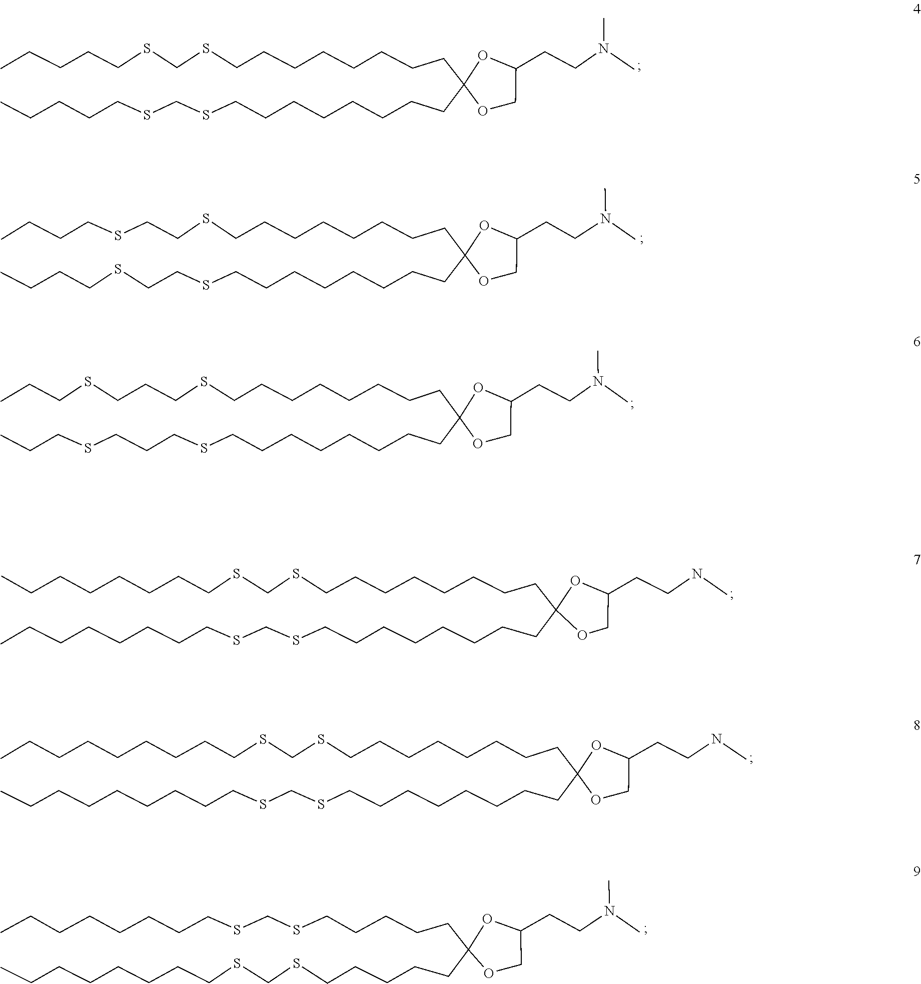

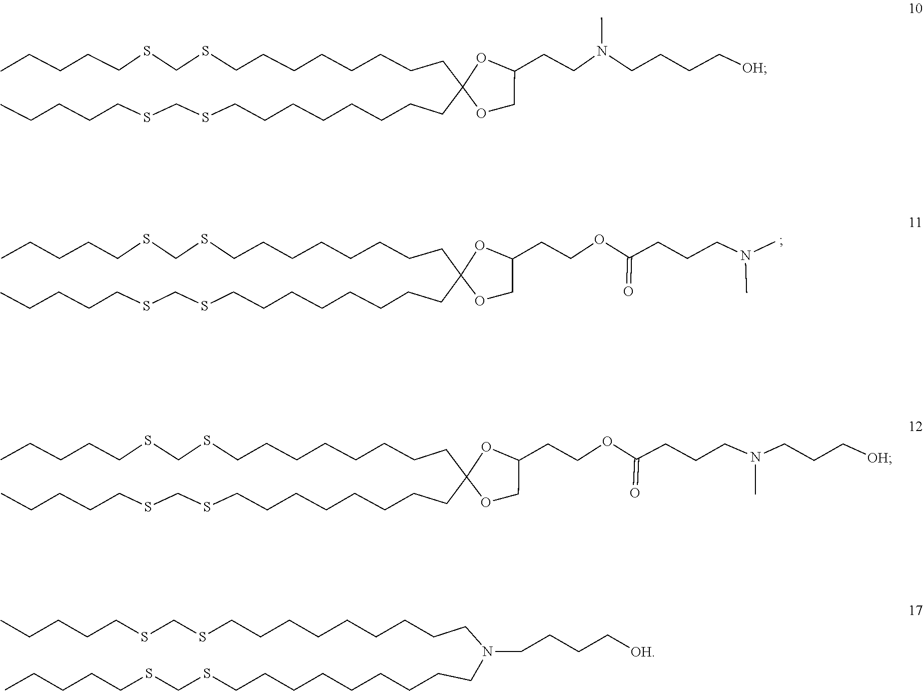

3. A lipid nanoparticle having a lipid or the salt thereof as defined in claim 1 and incorporating a therapeutic agent or prodrug therein.

4. The lipid nanoparticle of claim 3, wherein the therapeutic agent is a nucleic acid.

5. The lipid nanoparticle of claim 4, wherein the nucleic acid is mRNA.

6. (canceled)

7. (canceled)

8. A method of treating a subject with a treatment targeted to a non-liver organ, tissue or cell comprising administering the lipid nanoparticle of claim 3 to the subject.
